(19) United States
(12) Patent Application Publication
(10) Pub. No.: US 2004/0209858 A1
(43) Pub. Date: Oct. 21, 2004

(54) Cycloalkylamides and their therapeutic applications

(76) Inventors: Youssef L. Bennani, Shaker Heights, OH (US); Steve A. Chamberlin, Waukegan, IL (US); Sanjay R. Chemburkar, Gurnee, IL (US); Jinhua Chen, Gurnee, IL (US); Michael J. Dart, Highland Park, IL (US); Ashok K. Gupta, Gurnee, IL (US); Lei Wang, Libertyville, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/691,167

(22) Filed: Oct. 22, 2003

Related U.S. Application Data
(60) Provisional application No. 60/420,211, filed on Oct. 22, 2002.

(51) Int. Cl. A61K 31/54; A61K 31/397; A61K 31/537; A61K 31/495; A61K 31/445; A61K 31/401; A61K 31/16

(52) U.S. Cl. 514/183; 514/210.17; 514/227.5; 514/237.5; 514/255.01; 514/317; 514/423; 514/613

(57) ABSTRACT

The present invention relates to the use of compounds of formula (I)

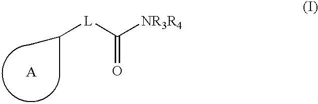

for the treatment of a variety of disorders including, but not limited to, epilepsy, bipolar disorder, psychiatric disorders, migraine, pain, neuroprotection, and movement disorders.

This application claims priority to U.S. Provisional Application Serial No. 60/420,211, filed Oct. 22, 2002.

### TECHNICAL FIELD

The present invention relates to cycloalkyl amides and acids, to the use of these compounds to treat epilepsy, bipolar disorder, psychiatric disorders, migraine, pain, movement disorders, and to the use of these compounds to provide neuroprotection, and to the preparation of these compounds.

### BACKGROUND OF THE INVENTION

Epilepsy is a common neurological disorder characterized by spontaneous recurrent seizures resulting from abnormal electrical discharges in the brain. It is a health problem that affects roughly 1% of the worldwide population (Loscher, W., Current status and future directions in the pharmacotherapy of epilepsy, Trends Pharmacol. Sci., 2002, 23 (3), 113-118). Epileptic seizures are divided into two major groups, partial or generalized. Partial (focal or local) seizures originate from one or more localized parts of the brain, whereas generalized seizures simultaneously emanate from both brain hemispheres. More than 40 distinct epilepsies have been identified and are characterized by a variety of factors including type of seizure, etiology, age of onset, severity, and EEG features (Commision on Classification and Terminology of the International League Against Epilepsy, Proposal for revised classification of epilepsies and epileptic syndromes, Epilepsia, 1989, 30 (4), 389-399). Epileptic disorders encompass a broad range of severities, extending from a mild and benign condition that readily responds to antiepileptic or anticonvulsant drug (AED) treatment, to a severe, debilitating and even life-threatening condition in which the recurrent seizures remain intractable to drug treatment.

Numerous drugs are now available for the symptomatic treatment of epilepsy. Among these are “first generation” AEDs such as phenytoin, carbamazepine, phenobarbital, and valproate. Several new AEDs or “second generation” drugs such as lamotrigine, topiramate, vigabatrin, felbamate, oxcarbazepine, tiagabine, gabapentin, zonisamide, and levetiracetam have entered the marketplace in the last 15 years (Perucca, E., Clinical pharmacology and therapeutic use of the new antiepileptic drugs, Fundamental & Clinical Pharmacology, 2001, 15, 405-417). Although the newer AEDs provide benefits, significant efficacy and safety issues remain (Schmidt, D., The clinical impact of new antiepileptic drugs after a decade of use in epilepsy, Epilepsy Res., 2002, 50(1-2), 21-32; Asconape, J. J., Some common issues in the use of antiepileptic drugs, Seminars in Neurology, 2002, 22(1), 27-39; and Wallace, S. J., Newer antiepileptic drugs: advantages and disadvantages, Brain & Development, 2001, 23, 277-283). For example, roughly one third of epileptic patients continue to have seizures. (Loscher, W. and Schmidt, D., New horizons in the development of antiepileptic drugs, Epilepsy Res., 2002 50 (1-2), 3-16). Thus, an urgent and unmet need exists for new AEDs with improved safety and efficacy.

The mechanisms of action of many AEDs are not well characterized, and for some, completely unknown. However, AEDs manage to strike a balance between inhibitory and excitatory mechanisms within the CNS, which ultimately can prevent seizures. At the cellular level, this antiseizure effect appears to be produced by several mechanisms that are generally divided into three main categories: modulation of voltage-gated ion channels (sodium, calcium, and potassium), indirect or direct enhancement of γ-aminobutyric acid (GABA)-mediated inhibitory neurotransmission, and inhibition of excitatory (particularly glutamate-mediated) neurotransmission (Kwan, P., Sills, G., Brodie, M. J., The mechanisms of action of commonly used antiepileptic drugs, Pharmacology & Therapeutics, 2001, 90, 21-34; Soderpalm, B., Anticonvulsants: aspects of their mechanisms of action, Eur. J. Pain, 2002, 6(Suppl A), 3-9). Many AEDs exert their actions through multiple mechanisms. In addition, numerous other less well-characterized mechanisms might also be operative and contribute to the biological activity of these drugs.

Several drugs developed initially as AEDs exhibit beneficial effects in a number of common neurological and psychiatric disorders including bipolar disorders, migraine, neuropathic pain, and movement disorders (Beghi, E., The use of anticonvulsants in neurological conditions other than epilepsy, CNS Drugs, 1999, 11 (1), 61-82). The spectrum of uses for AEDs in psychiatric disorders continues to expand. It has been reported that one third of patients currently taking AEDs do so for the treatment of diverse CNS disorders other than epilepsy (Lopes da Silva, F., Post, R. M., Evaluation and prediction of effects of antiepileptic drugs in a variety of other CNS disorders, Epilepsy Research, 2002, 50(1-2), 191-193). Given the increasingly diverse range of clinical utility being recognized with AEDs, it is likely that new chemical entities, which display broad-spectrum anticonvulsant activity, may also show beneficial effects for the treatment of a variety of neurological and psychiatric disorders.

Several AEDs are used clinically to treat the various aspects of bipolar disorder, which is a chronic, cyclic disease characterized by disruptive mood swings from mania to depression. It is a chronic disorder that affects more than 1% of the US population. Carbamazepine was the first AED utilized to treat bipolar disorder (Brambilla, P., Barale, F., Soares, J. C., Perspectives on the use of anticonvulsants in the treatment of bipolar disorder, International Journal of Neuropsychopharmacology, 2001, 4, 421-446). Valproate has more recently emerged and now competes with lithium as a first-line treatment for patients with bipolar disorder, in particular the manic episodes associated with this illness (Angel, I. and Horovitz, T., Bipolar disorder and valproic acid, Current Opinion in Central & Peripheral Nervous System Investigational Drugs (1999), 1(4), 466-469; Bowden, C. L., Brugger, A. M., Swann, A. C., Calabrese, J. R., Janicak, P. G., Petty, F., Dilsaver, S. C., Davis, J. M., Rush, A. J., Small, J. G., Garza-Trevino, E. S., Risch S. C., Goodnick, P. J., Morris, D. D., Efficacy of divalproex vs lithium and placebo in the treatment of mania. The Depakote Mania Study Group, JAMA, 1994, 271(12), 918-24). Lamotrigine has shown beneficial effects in the treatment of bipolar depression (Muzina, D. J., El-Sayegh, S., Calabrese, J. R., Antiepileptic drugs in psychiatry-focus on randomized controlled trial, Epilepsy Research, 2002, 50 (1-2), 195-202; Calabrese, J. R., Shelton, M. D., Rapport, D. J., Kimmel, S. E., Bipolar disorders and the effectiveness of novel anticonvulsants, J. Clin. Psychiatry, 2002, 63 (suppl 3), 5-9).

In addition to bipolar disorder, a number of neuropsychiatric syndromes and disorders may be treated with AEDs (Bialer, M., Johannessen, S. I., Kupferberg, H. J., Levy, R. H., Loiseau, P., Perucca, E., Progress report on new antiepileptic drugs: a summary of the sixth eilat conference (EILAT VI), Epilepsy Res. 2002, 51, 31-71; Fountain, N. B., Dreifuss, F. E., The future of valproate. In: Valproate., Loscher W., Editor. 1999, Birkhauser Verlag, Boston). Such psychiatric disorders include: anxiety and panic disorders, post-traumatic stress disorder, schizophrenia, episodic dyscontrol, substance-abuse-related disorders, impulse control disorders, general agitation associated with a variety of psychiatric disorders and dementias, and behavioral disorders associated with autism.

Migraine is defined as a periodically occurring vascular headache characterized by pain in the head (usually unilateral), nausea and vomiting, photophobia, phonophobia, vertigo and general weakness. It is associated with episodic as well as long-term disability and suffering. Migraine is the most common type of vascular headache and affects as much as 15% of the world‘s population (Krymchantowski, A. V., Bigal, M. E., Moreira, P. E., New and emerging prophylactic agents for migraine, CNS Drugs, 2002, 16 (9), 611-634). Several AEDs have been shown to be effective in the prevention of migraine including valproate, lamotrigine, gabapentin, and topiramate (Wheeler, S. D., Antiepileptic drug therapy in migraine headache, Current Treatment Options in Neurology, 2002, 4, 383-394; Krymchantowski, A. V., Bigal, M. E., Moreira, P. E., New and emerging prophylactic agents for migraine, CNS Drugs, 2002, 16 (9), 611-634). Many AEDs act by attenuating cellular hyperexcitability and providing a balance between GABAergic inhibition and excitatory amino acid-mediated neuronal excitation, factors that may play a role in the pathophysiology of migraines.

Pain is a common symptom of disease and a frequent complaint with which patients present to physicians. Pain is commonly segmented by duration (acute vs. chronic), intensity (mild, moderate, and severe), and type of pain (nociceptive vs. neuropthic). Neuropathic pain encompasses a wide range of pain syndromes of diverse etiologies and is characterized by a neuronal hyperexcitablility in damaged areas of the nervous system. Diabetic neuropathy, cancer neuropathy, and HIV pain are the most commonly diagnosed types of neuropathic pain. Neuropathic pain also afflicts a significant number of patients suffering from a wide range of other disorders such as trigeminal neuralgia, post-herpetic neuralgia, traumatic neuralgia, phantom limb, as well as numerous other painful disorders of ill-defined or unknown origin. Patients generally respond poorly to traditional pain therapeutic approaches and new drugs with improved efficacy, tolerability, and safety are needed.

Carbamazepine was the first AED examined in controlled trials for neuropathic pain and the results support its use in the treatment of paroxysmal attacks in trigeminal neuralgia, post-herpetic neuralgia, and diabetic neuropathy (Jensen, T. S., Anticonvulsants in neuropathic pain: rationale and clinical evidence, Eur. J. Pain, 2002, 6 (suppl A), 61-68). Among the AEDs examined in controlled trials, gabapentin has clearly demonstrated analgesic effects in treating postherpetic neuralgia and painful diabetic neuropathy (Tremont-Lukats, I. W., Megeff, C., Backonja, M.-M., Anticonvulsants for neuropathic pain syndromes: mechanisms of action and place in therapy, Drugs, 60 (5), 1029-1052). Lamotrigine has demonstrated efficacy in relieving pain in patients with trigeminal neuralgia refractory to other treatments (Backonja, M.-M., Anticonvulsants (antineuropathics) for neuropathic pain syndromes, Clin. J. Pain, 2000, 16, S67-S72). Pregabalin, a follow-on compound to gabapentin, has shown efficacy in clinical trials for diabetic neuropthy. In addition, numerous AEDs display antinociceptive, antiallodynic, or antihyperalgesic activity in animal models relevant to a variety of pain states. Therefore, the potential exists for new AEDs to benefit patients suffering from pain.

AEDs have also been used clinically to treat a variety of movement disorders (Magnus, L., Nonepileptic uses of gabapentin, Epilepsia, 1999, 40 (suppl 6), S66-S72; Fountain, N. B., Dreifuss, F. E., The future of valproate. In: Valproate., Löscher W., Editor. 1999, Birkhauser Verlag, Boston; Cutter, N., Scott, D. D., Johnson, J. C., Whiteneck, G., Gabapentin effect on spacticity in multiple sclerosis, a placebo-controlled, randomized trial, 2000, 81, 164-169), and shown positive effects in animal models of movement disorders (Löscher W., Richter, A., Piracetam and levetiracetam, two pyrrolidone derivatives, exert antidystonic activity in a hamster model of paroxysmal dystonia, Eur. J. Pharmacol., 2000, 391, 251-254). Movement disorders include restless leg syndrome, essential tremor, acquired nystagmus, post-anoxic myoclonus, spinal myoclonus, spasticity, chorea, and dystonia.

Many AEDs have demonstrated some evidence of neuroprotective activity in a variety of ischemia models (Pitkanen, A., Efficacy of current antiepileptics to prevent neurodegeneration in epilepsy models, Epilepsy Research, 2002, 50, 141-160). These neuroprotective effects indicate that AEDs could be useful in the treatment of stroke, in mitigating brain damage after recovery from cardiac arrest, and in preventing epileptogenesis.

The present invention relates to compounds that are anticonvulsants and therefore can be used to treat a variety of indications including, but not limited to, epilepsy, bipolar disorder, psychiatric disorders, migraine, pain, movement disorders, and to provide neuroprotection.

### SUMMARY OF THE INVENTION

In its principle embodiment, the present invention relates to a method of treating migraine, epilepsy, or bipolar disorder in a mammal, particularly in a human, comprising administering to a mammal a therapeutically effective amount of a compound of formula (I)

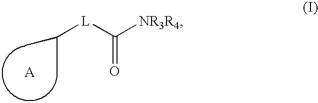

or a pharmaceutically acceptable prodrug thereof, wherein

A is cycloalkyl or bridged cycloalkyl;

L is a single bond or alkylene;

R₃ and R₄ are independently hydrogen, alkenyl, alkyl, alkynyl, alkoxycarbonylalkyl, aryl, arylalkyl, carboxyalkyl, cycloalkyl, cycloalkylalkyl, heterocycle, heterocyclealkyl, hydroxyalkyl, (NR₅R₆)alkyl, (NR₅R₆)carbonylalkyl, or

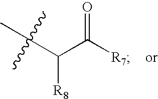

R₃ and R₄ taken together with the nitrogen atom to which they are attached form a heterocycle wherein the heterocycle is azepanyl, azetidinyl, aziridinyl, morpholinyl, piperazinyl, piperidinyl, pyrrolidinyl, or thiomorpholinyl;

R₅ and R₆ are independently hydrogen, alkenyl, alkyl, alkynyl, alkoxycarbonylalkyl, aryl, arylalkyl, cycloalkyl, cycloalkylalkyl, heterocycle, heterocyclealkyl, or hydroxyalkyl;

R₇ is alkoxy, alkyl, hydroxy, or —NR₅R₆;

R₈ is alkenyl, alkoxyalkyl, alkoxycarbonylalkyl, alkylthioalkyl, alkynyl, aryl, arylalkyl, carboxyalkyl, cycloalkyl, cycloalkylalkyl, heterocycle, heterocyclealkyl, hydroxyalkyl, mercaptoalkyl, (NR₅R₆)alkyl, (NR₅R₆)carbonylalkyl, or —(CH₂)nNHC(═NH)NH₂; and

n is an integer from 1 to 6;

provided that the compound of formula (I) is other than cyclohexanecarboxamide.

### DETAILED DESCRIPTION OF THE INVENTION

All patents, patent applications, and literature references cited in the specification are herein incorporated by reference in their entirety.

In its principle embodiment, the present invention relates to a method of treating migraine, epilepsy, or bipolar disorder in a mammal, particularly in a human, comprising administering to a mammal a therapeutically effective amount of a compound of formula (I)

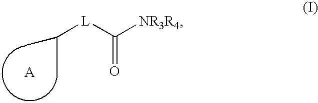

or a pharmaceutically acceptable prodrug thereof, wherein

A is cycloalkyl or bridged cycloalkyl;

L is a single bond or alkylene;

R₃ and R₄ are independently hydrogen, alkenyl, alkyl, alkynyl, alkoxycarbonylalkyl, aryl, arylalkyl, carboxyalkyl, cycloalkyl, cycloalkylalkyl, heterocycle, heterocyclealkyl, hydroxyalkyl, (NR₅R₆)alkyl, (NR₅R₆)carbonylalkyl, or

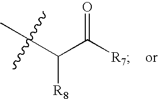

R₃ and R₄ taken together with the nitrogen atom to which they are attached form a heterocycle wherein the heterocycle is azepanyl, azetidinyl, aziridinyl, morpholinyl, piperazinyl, piperidinyl, pyrrolidinyl, or thiomorpholinyl;

R₅ and R₆ are independently hydrogen, alkenyl, alkyl, alkynyl, alkoxycarbonylalkyl, aryl, arylalkyl, cycloalkyl, cycloalkylalkyl, heterocycle, heterocyclealkyl, or hydroxyalkyl;

R₇ is alkoxy, alkyl, hydroxy, or —NR₅R₆;

R₈ is alkenyl, alkoxyalkyl, alkoxycarbonylalkyl, alkylthioalkyl, alkynyl, aryl, arylalkyl, carboxyalkyl, cycloalkyl, cycloalkylalkyl, heterocycle, heterocyclealkyl, hydroxyalkyl, mercaptoalkyl, (NR₅R₆)alkyl, (NR₅R₆)carbonylalkyl, or —(CH₂)ₙNHC(═NH)NH₂; and

n is an integer from 1 to 6;

provided that the compound of formula (I) is other than cyclohexanecarboxamide.

In another embodiment, the present invention relates to a method of treating epilepsy, migraine, or bipolar disorder in a mammal comprising administering to a mammal a therapeutically effective amount of a compound of formula (I) wherein A is cycloalkyl; and L, R₃ and R₄ are as defined in formula (I).

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl substituted with 1, 2, or 3 alkyl groups; L is a single bond; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; R₃ is hydrogen; and R₄ is alkyl, wherein a preferred alkyl is methyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl substituted with 1, 2, or 3 alkyl groups; L is a single bond; R₃ is hydrogen; and R₄ is alkyl, wherein a preferred alkyl is methyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is 3,5-dimethylcyclohexyl; L is a single bond; R₃ is hydrogen; and R₄ is alkyl, wherein a preferred alkyl is methyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopentyl optionally substituted with 1, 2, or 3 alkyl groups; L is a single bond; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopentyl; L is a single bond; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl substituted with 1, 2, or 3 alkyl groups; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl substituted with 2 alkyl groups; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is 3,5-dimethylcyclohexyl; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is 3,5-dimethylcyclohexyl; L is a single bond; R₃ is hydrogen; and R₄ is (NR₅R₆)carbonylalkyl wherein the (NR₅R₆)carbonylalkyl is 2-amino-2-oxoethyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopentyl optionally substituted with 1, 2, or 3 alkyl groups; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopentyl; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; R₃ is hydrogen; R₄ is

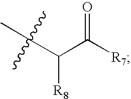

and R₇ and R₈ are as defined in formula (I).

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; R₃ is hydrogen; R₄ is

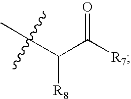

R₇ is NR₅R₆; R₅ and R₆ are hydrogen; and R₈ is heterocycle wherein the heterocycle is imidazolyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; R₃ is hydrogen; and R₄ is carboxyalkyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is a single bond; R₃ is hydrogen; and R₄ is hydroxyalkyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl substituted with 1, 2, or 3 alkyl groups; L is a single bond; R₃ is hydrogen; and R₄ is hydroxyalkyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is 3,5-dimethylcyclohexyl; L is a single bond; R₃ is hydrogen; and R₄ is hydroxyalkyl wherein the hydroxyalkyl is 2-hydroxypropyl.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopropyl optionally substituted with 1, or 2 cyclopropyl groups; L is a single bond; R₃ is hydrogen; R₄ is hydrogen or (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopropyl substituted with 1, or 2 cyclopropyl groups; L is a single bond; R₃ is hydrogen; R₄ is hydrogen or (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is alkylene; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is alkylene wherein the alkylene is CH₂; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopentyl optionally substituted with 1, 2, or 3 alkyl groups; L is alkylene wherein the alkylene is CH₂; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclohexyl optionally substituted with 1, 2, or 3 alkyl groups; L is alkylene wherein the alkylene is CH₂; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is cycloalkyl wherein the cycloalkyl is cyclopentyl optionally substituted with 1, 2, or 3 alkyl groups; L is alkylene wherein the alkylene is CH₂; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is bridged cycloalkyl; and L, R₃ and R₄ are as defined in formula (I).

In another embodiment, the present invention relates to a method of treating epilepsy, migraine, or bipolar disorder in a mammal comprising administering to a mammal a therapeutically effective amount of a compound of formula (I) wherein A is bridged cycloalkyl wherein the bridged cycloalkyl is adamantane, bicyclo[2.2.1]heptane, or octahydro-2,5-methanopentalene; L is a single bond; and R₃ and R₄ are hydrogen.

In another embodiment, the present invention relates to a method of treating epilepsy, migraine, or bipolar disorder in a mammal comprising administering to a mammal a therapeutically effective amount of a compound of formula (I) wherein A is bridged cycloalkyl wherein the bridged cycloalkyl is adamantane, bicyclo[2.2.1]heptane, or octahydro-2,5-methanopentalene; L is a single bond; R₃ is hydrogen; R₄ is (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is bridged cycloalkyl wherein the bridged cycloalkyl is adamantane, bicyclo[2.2.1]heptane, or octahydro-2,5-methanopentalene; L is alkylene; R₃ is hydrogen; R₄ is hydrogen or (NR₅R₆)carbonylalkyl; and R₅ and R₆ are hydrogen.

In another embodiment, the present invention relates to a method of treating epilepsy, migraine, or bipolar disorder in a mammal comprising administering to a mammal a therapeutically effective amount of a compound of formula (I) wherein A is bridged cycloalkyl wherein the bridged cycloalkyl is adamantane, bicyclo[2.2.1]heptane, or octahydro-2,5-methanopentalene; L is alkylene wherein the alkylene is CH₂; R₃ is hydrogen; R₄ is hydrogen or (NR₅R₆)carbonylalkyl; and R₅ and R₆ are hydrogen.

In another embodiment, the present invention relates to a method of treating migraine, epilepsy, or bipolar disorder, comprising administering to a mammal a therapeutically effective amount of (cis) (3R,5S)—N-(2-amino-2-oxoethyl)-3,5-dimethylcyclohexanecarboxamide.

In another embodiment, the present invention relates to a method of treating a psychiatric disorder, pain, or a movement disorder, comprising administering to a mammal a therapeutically effective amount of a compound of formula (I).

In another embodiment, the present invention relates to a method of treating a psychiatric disorder, pain, or a movement disorder, comprising administering to a mammal a therapeutically effective amount of (cis) (3R,5S)—N-(2-amino-2-oxoethyl)-3,5-dimethylcyclohexanecarboxamide.

In another embodiment, the present invention relates to a method of providing neuroprotection in a mammal comprising administering to a mammal a therapeutically effective amount of a compound of formula (I).

In another embodiment, the present invention relates to a method of providing neuroprotection in a mammal comprising administering to a mammal a therapeutically effective amount of (cis) (3R,5S)—N-(2-amino-2-oxoethyl)-3,5-dimethylcyclohexanecarboxamide.

Representative compounds of formula (I) include, but are not limited to:

2-(3-methylcyclohexyl)acetamide;

N-(2-amino-2-oxoethyl)-2-(3-methylcyclohexyl)acetamide;

N-(2-amino-2-oxoethyl)-4-methylcyclohexanecarboxamide;

N-(2-amino-2-oxoethyl)-3-methylcyclohexanecarboxamide;

(1S,2R,5S)—N-(2-amino-2-oxoethyl)-2,5-dimethylcyclohexanecarboxamide;

N-(2-amino-2-oxoethyl)-2,3-dimethylcyclohexanecarboxamide;

N-(2-amino-2-oxoethyl)-2-methylcyclohexanecarboxamide;

N-(2-amino-2-oxoethyl)-3,4-dimethylcyclohexanecarboxamide;

N-(2-amino-2-oxoethyl)-4-isopropylcyclohexanecarboxamide;

N-(2-amino-2-oxoethyl)-4-tert-butylcyclohexanecarboxamide;

N-(2-amino-2-oxoethyl)bicyclo[2.2.1]heptane-2-carboxamide;

N-(2-amino-2-oxoethyl)-1-adamantanecarboxamide;

N-(2-amino-2-oxoethyl)hexahydro-2,5-methanopentalene-3a(1H)-carboxamide;

N-(2-amino-2-oxoethyl)-2,4-dimethylcyclohexanecarboxamide;

(cis) (3R,5S)—N-[(1S)-2-amino-1-methyl-2-oxoethyl]-3,5-dimethylcyclohexanecarboxamide;

(cis) (3R,5S)—N-(3-amino-3-oxopropyl)-3,5-dimethylcyclohexanecarboxamide;

(cis) (3R,5S)—N-[(1R)-2-amino-1-methyl-2-oxoethyl]-3,5-dimethylcyclohexanecarboxamide;

(cis) (3R,5S)—N-[(1S)-2-amino-1-(1H-imidazol-4-ylmethyl)-2-oxoethyl]-3,5-dimethylcyclohexanecarboxamide;

(cis) (3R,5S)—N-[(1S)-1-(aminocarbonyl)-2-methylpropyl]-3,5-dimethylcyclohexanecarboxamide;

(cis) (2S)-2-({[(3R,5S)-3,5-dimethylcyclohexyl]carbonyl}amino)propanoic acid;

2-cyclopentylacetamide;

N-(2-amino-2-oxoethyl)-2-cyclopentylacetamide;

2-cyclohexylacetamide;

N-(2-amino-2-oxoethyl)-2-cyclohexylacetamide;

2-(4-methylcyclohexyl)acetamide;

N-(2-amino-2-oxoethyl)-2-(4-methylcyclohexyl)acetamide;

424432 2-(2-methylcyclohexyl)acetamide;

N-(2-amino-2-oxoethyl)-2-(2-methylcyclohexyl)acetamide;

2-(5-isopropyl-2-methylcyclohexyl)acetamide;

N-(2-amino-2-oxoethyl)-2-(5-isopropyl-2-methylcyclohexyl)acetamide;

2-(4-tert-butylcyclohexyl)acetamide;

N-(2-amino-2-oxoethyl)-2-(4-tert-butylcyclohexyl)acetamide;

2-bicyclo[2.2.1]hept-2-ylacetamide;

N-(2-amino-2-oxoethyl)-2-bicyclo[2.2.1]hept-2-ylacetamide;

2-(1-adamantyl)acetamide;

2-(1-adamantyl)-N-(2-amino-2-oxoethyl)acetamide;

2-(4,4-dimethylcyclohexyl)acetamide;

N-(2-amino-2-oxoethyl)-2-(4,4-dimethylcyclohexyl)acetamide;

1,1′:1′,1″-ter(cyclopropane)-2′-carboxamide;

N-(2-amino-2-oxoethyl)-1,1′:1′,1″-ter(cyclopropane)-2′-carboxamide;

(cis) (3R,5S)-3,5-dimethylcyclohexanecarboxamide;

2,3-dimethylcyclohexanecarboxamide;

4-methylcyclohexanecarboxamide;

3-methylcyclohexanecarboxamide;

2-methylcyclohexanecarboxamide;

cyclopentanecarboxamide;

2,5-dimethylcyclohexanecarboxamide;

3,4-dimethylcyclohexanecarboxamide;

4-isopropylcyclohexanecarboxamide;

4-tert-butylcyclohexanecarboxamide;

bicyclo[2.2.1]heptane-2-carboxamide;

1-adamantanecarboxamide;

hexahydro-2,5-methanopentalene-3a(1H)-carboxamide;

2,4-dimethylcyclohexanecarboxamide;

(cis) (2R,6S)-2,4,6-trimethylcyclohexanecarboxamide;

N-(2-amino-2-oxoethyl)cyclopentanecarboxamide;

N-(2-amino-2-oxoethyl)-2,5-dimethylcyclohexanecarboxamide;

(cis) (2R,6S)—N-(2-amino-2-oxoethyl)-2,4,6-trimethylcyclohexanecarboxamide;

(cis) (3R,5S)—N-[(2R)-2-hydroxypropyl]-3,5-dimethylcyclohexanecarboxamide;

(cis) (3R,5S)—N-[(2S)-2-hydroxypropyl]-3,5-dimethylcyclohexanecarboxamide;

and

(3R,5S)—N,3,5-trimethylcyclohexanecarboxamide or pharmaceutically acceptable prodrugs thereof.

A preferred compound of formula (I) is (cis) (3R,5S)—N-(2-amino-2-oxoethyl)-3,5-dimethylcyclohexanecarboxamide or a pharmaceutically acceptable prodrug thereof.

In another embodiment, the present invention relates to a compound of formula (II)

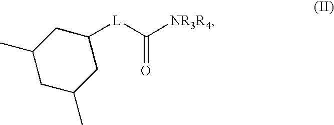

or a pharmaceutically acceptable prodrug thereof, wherein

L is a single bond or alkylene;

R₃ is hydrogen or alkyl;

R₄ is alkenyl, alkyl, alkynyl, alkoxycarbonylalkyl, aryl, arylalkyl, carboxyalkyl, cycloalkyl, cycloalkylalkyl, heterocycle, heterocyclealkyl, hydroxyalkyl, (NR₅R₆)alkyl, (NR₅R₆)carbonylalkyl, or

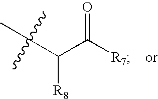

R₃ and R₄ taken together with the nitrogen atom to which they are attached form a heterocycle wherein the heterocycle is azepanyl, azetidinyl, aziridinyl, morpholinyl, piperazinyl, piperidinyl, pyrrolidinyl, or thiomorpholinyl;

R₅ and R₆ are independently hydrogen, alkenyl, alkyl, alkynyl, alkoxycarbonylalkyl, aryl, arylalkyl, cycloalkyl, cycloalkylalkyl, heterocycle, heterocyclealkyl, or hydroxyalkyl;

R₇ is alkoxy, alkyl, hydroxy, or —NR₅R₆;

R₈ is alkenyl, alkoxyalkyl, alkoxycarbonylalkyl, alkylthioalkyl, alkynyl, aryl, arylalkyl, carboxyalkyl, cycloalkyl, cycloalkylalkyl, heterocycle, heterocyclealkyl, hydroxyalkyl, mercaptoalkyl, (NR₅R₆)alkyl, (NR₅R₆)carbonylalkyl, or —(CH₂)ₙNHC(═NH)NH₂; and

n is an integer from 1 to 6.

In another embodiment, the present invention relates to a compound of formula (II) wherein L is a single bond; R₃ is hydrogen; and R₄ is alkyl, wherein a preferred alkyl is methyl.

In another embodiment, the present invention relates to a compound of formula (II) wherein R₃ is hydrogen; R₄ is (NR₅R₆)carbonylalkyl; and L, R₅ and R₆ are as defined in formula (II).

In another embodiment, the present invention relates to a compound of formula (II) wherein L is a single bond; R₃ is hydrogen; R₄ is (NR₅R₆)carbonylalkyl; and R₅ and R₆ are hydrogen.

In another embodiment, the present invention relates to a compound of formula (II) wherein L is a single bond; R₃ is hydrogen; R₄ is (NR₅R₆)carbonylalkyl wherein the (NR₅R₆)carbonylalkyl is 2-amino-2-oxoethyl; and R₅ and R₆ are hydrogen.

In another embodiment, the present invention relates to a compound of formula (II) wherein L is a single bond; R₃ is hydrogen; and R₄ is carboxyalkyl.

In another embodiment, the present invention relates to a compound of formula (II) wherein L is a single bond; R₃ is hydrogen; and R₄ is

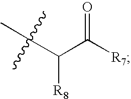

and R₇ and R₈ are as defined in formula (II).

In another embodiment, the present invention relates to a compound of formula (II) wherein L is a single bond; R₃ is hydrogen; R₄ is

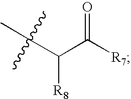

R₈ is heterocycle; and R₇ is as defined in formula (II).

In another embodiment, the present invention relates to a compound of formula (II) wherein L is a single bond; R₃ is hydrogen; R₄ is

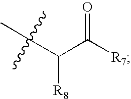

R₇ is —NR₅R₆; R₅ and R₆ are independently hydrogen or alkyl; and R₈ is heterocycle wherein the heterocycle is imidazolyl.

In another embodiment, the present invention relates to a compound of formula (II) wherein L is a single bond; R₃ is hydrogen; and R₄ is hydroxyalkyl.

In another embodiment, the present invention relates to a compound of formula (II) wherein L is a single bond; R₃ is hydrogen; R₄ is hydroxyalkyl wherein the hydroxyalkyl is 2-hydroxypropyl; and R₅ and R₆ are hydrogen.

Rperesentative compounds of formula (II) include, but are not limited to:

(cis) (3R,5S)—N-[(1S)-2-amino-1-methyl-2-oxoethyl]-3,5-dimethylcyclohexanecarboxamide;

(cis) (3R,5S)—N-(3-amino-3-oxopropyl)-3,5-dimethylcyclohexanecarboxamide;

(cis) (3R,5S)—N-[(1R)-2-amino-1-methyl-2-oxoethyl]-3,5-dimethylcyclohexanecarboxamide;

(cis) (3R,5S)—N-[(1S)-2-amino-1-(1H-imidazol-4-ylmethyl)-2-oxoethyl]-3,5-dimethylcyclohexanecarboxamide;

(cis) (3R,5S)—N-[(1S)-1-(aminocarbonyl)-2-methylpropyl]-3,5-dimethylcyclohexanecarboxamide;

(cis) (2S)-2-({[(3R,5S)-3,5-dimethylcyclohexyl]carbonyl}amino)propanoic acid;

(cis) (3R,5S)—N-[(2R)-2-hydroxypropyl]-3,5-dimethylcyclohexanecarboxamide; and

(cis) (3R,5S)—N-[(2S)-2-hydroxypropyl]-3,5-dimethylcyclohexanecarboxamide; or pharmaceutically acceptable prodrugs thereof.

A preferred compound of formula (II) is (cis) (3R,5S)—N-(2-amino-2-oxoethyl)-3,5-dimethylcyclohexanecarboxamide or pharmaceutically acceptable prodrugs thereof.

Definition of Terms

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited, methylthiomethyl and 2-(ethylthio)ethyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a monocyclic-ring system, or a bicyclic-, or a tricyclic-fused ring system wherein one or more of the fused rings are aromatic. Representative examples of aryl include, but are not limited to, anthracenyl, azulenyl, fluorenyl, 2,3-dihydroindenyl, indenyl, naphthyl, phenyl, and tetrahydronaphthyl.

The aryl groups of this invention can be optionally substituted with 1, 2, or 3 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfonyl, alkylthio, alkylthioalkyl, alkynyl, carboxy, carboxyalkyl, cyano, cyanoalkyl, ethylenedioxy, formyl, haloalkoxy, haloalkyl, halogen, hydroxy, hydroxyalkyl, mercapto, methylenedioxy, nitro, —NR_{D}R_{E}, and (NR_{D}R_{E})carbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “bridged cycloalkyl” as used herein, means a saturated bicyclic or tricyclic ring system. Bicyclic ring systems are exemplified by a cycloalkyl group, as defined herein, in which two non-adjacent carbon atoms of the cycloalkyl group are linked by an alkylene bridge of between one and three carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The bridged cycloalkyl groups of the present invention are optionally substituted with 1, 2, 3, or 4 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, oxo, mercapto, —NR_{D}R_{E}, and (NR_{D}R_{E})carbonyl.

The term “carbonyl” as used herein, means a —C(O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl” as used herein, means a monocyclic ring system exemplified by a saturated cyclic hydrocarbon group containing from 3 to 8 carbon atoms. Examples of cycloalkyl include cyclopropyl, cyclobutyl, cyclopentyl, cyclohexyl, cycloheptyl, and cyclooctyl.

The cycloalkyl groups of the present invention are optionally substituted with 1, 2, 3, or 4 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, oxo, mercapto, —NR_{D}R_{E}, and (NR_{D}R_{E})carbonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “ethylenedioxy” as used herein, means a —O(CH₂)₂O— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl” as used herein, means a —C(O)H group.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic” as used herein, means a monocyclic, bicyclic, or tricyclic ring system. Monocyclic ring systems are exemplified by any 3- or 4-membered ring containing a heteroatom independently selected from the group consisting of oxygen, nitrogen and sulfur; or a 5-, 6- or 7-membered ring containing one, two or three heteroatoms wherein the heteroatoms are independently selected from the group consisting of nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzofuranyl, isobenzothienyl, isoindolyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl.

The heterocycles of this invention are optionally substituted with 1, 2,or 3 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, ethylenedioxy, formyl, haloalkoxy, haloalkyl, halogen, hydroxy, hydroxyalkyl, mercapto, methylenedioxy, nitro, oxo, —NR_{D}R_{E} and (NR_{D}R_{E})carbonyl.

The term “heterocyclealkyl” as used herein, means a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, hydroxymethyl, 2-hydroxyethyl, 2-hydroxypropyl, 3-hydroxypropyl, 2,3-dihydroxypentyl, and 2-ethyl-4-hydroxyheptyl.

The term “mercapto” as used herein, means a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “methylenedioxy” as used herein, means a —OCH₂O— group wherein the oxygen atoms of the methylenedioxy are attached to the parent molecular moiety through two adjacent carbon atoms.

The term “nitro” as used herein, means a —NO₂ group.

The term “—NR_{D}R_{E}” as used herein, means two groups, R_{D} and R_{E}, which are appended to the parent molecular moiety through a nitrogen atom. R_{D} and R_{E} are independently hydrogen, alkenyl, alkoxycarbonyl, alkoxysulfonyl, alkyl, alkylcarbonyl, alkylsulfonyl, alkynyl, or formyl. Representative examples of—NR_{D}R_{E} include, but are not limited to, amino, acetylamino, methylamino, dimethylamino, ethylamino, ethylmethylamino, benzylamino, methoxysulfonylamino, methylsulfonylamino, ethoxycarbonylamino, and tert-butoxycarbonylamino.

The term “(NR_{D}R_{E})carbonyl” as used herein, means a —NR_{D}R_{E} group, as defined herein, appended to the parent molecular moiety through a carbonyl group, as defined herein. Representative examples of (NR_{D}R_{E})carbonyl include, but are not limited to, aminocarbonyl, (methylamino)carbonyl, (dimethylamino)carbonyl and (ethylmethylamino)carbonyl.

The term “(NR₅R₆)alkyl” as used herein, means a —NR₅R₆ group, as defined herein, appended to the parent molecular moiety through an alkyl group, as defined herein. Representative examples of (NR₅R₆) alkyl include, but are not limited to, 2-aminoethyl, 2-(dimethylamino)ethyl, and 3-aminopropyl.

The term “(NR₅R₆)carbonyl” as used herein, means a —NR₅R₆ group, as defined herein, appended to the parent molecular moiety through a carbonyl group, as defined herein. Representative examples of (NR₅R₆)carbonyl include, but are not limited to, aminocarbonyl, (methylamino)carbonyl, (dimethylamino)carbonyl, and (ethylmethylamino)carbonyl.

The term “(NR₅R₆)carbonylalkyl” as used herein, means a (NR₅R₆)carbonyl group, as defined herein, appended to the parent molecular moiety through an alkyl group, as defined herein. Representative examples of (NR₅R₆)carbonylalkyl include, but are not limited to, 2-amino-2-oxoethyl, 2-methylamino-2-oxoethyl, and 2-dimethylamino-2-oxoethyl.

The term “oxo” as used herein, means a ═O moiety.

The term “sulfonyl” as used herein, means a —SO₂— group.

Compounds of the present invention were named by ACD/ChemSketch version 5.0 (developed by Advanced Chemistry Development, Inc., Toronto, ON, Canada) or were given names consistent with ACD nomenclature.

Compounds of the present invention can exist as stereoisomers, wherein asymmetric or chiral centers are present. Stereoisomers are designated “R” or “S,” depending on the configuration of substituents around the chiral carbon atom. The terms “R” and “S” used herein are configurations as defined in IUPAC 1974 Recommendations for Section E, Fundamental Stereochemistry, Pure Appl. Chem., (1976), 45: 13-30. The present invention contemplates various stereoisomers and mixtures thereof and are specifically included within the scope of this invention. Stereoisomers include enantiomers, diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

### Abbreviations

Abbreviations which have been used in the descriptions of the schemes and the examples that follow are: DMSO for dimethylsulfoxide; TEA for triethylamine; Aldrich for Aldrich Chemical Company located in Milwaukee, Wis.; Acros for Acros Organics located in Morris Plains, N.J.; Lancaster for Lancaster Synthesis located in Windham, N.H.; and Bachem for Bachem Bioscience located in King of Prussia, Pa.

### Preparation of Compounds of The Invention

The compounds of the present invention can be prepared by a variety of synthetic routes. Representative procedures are shown in Scheme 1.

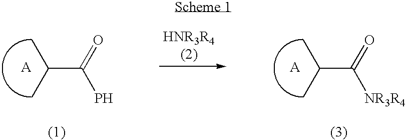

Amides of general formula (3), wherein A, R₃, and R₄ are as defined in formula (I), can be prepared as described in Scheme 1. Acids of general formula (1), purchased or prepared using chemistry known to those of ordinary skill in the art, can be treated with amines of general formula (2) and a coupling reagent including, but not limited to, 1,1′-carbonyldiimidazole (CDI), 1,1′-thiocarbonyldiimidazole, 1,3-dicyclohexylcarbodiimide, 1-ethyl-3-[3-(dimethylamino)propyl]carbodiimide hydrochloride, or thionylchloride, to provide amides of general formula (3).

### EXAMPLE 1

### (cis) (3R,5S)—N-(2-amino-2-oxoethyl)-3,5-dimethylcyclohexanecarboxamide

### EXAMPLE 1A

### (cis) (3R,5S)-3,5-dimethylcyclohexanecarboxylic acid

3,5-Dimethyl benzoic acid (500 g, 3.33 mol, purchased from Acros) and platinum(IV) oxide (25 g, 0.11 mol, purchased from Aldrich) were combined in acetic acid (3.5 L) at room temperature and agitated under 400 psi of hydrogen for about 45 minutes. The mixture was filtered and the filtrate was diluted with H₂O(21 L) resulting in formation of a precipitate. The solid was collected by filtration (H₂O wash) and dried to provide the title compound (464 g, 89% yield). ¹H NMR (CDCl₃, 300 MHz) δ 2.60(m,1H), 1.95(m, 2H), 1.67(m, 1H), 1.40-1.54(m, 2H), 0.96-1.07(m, 2H), 0.91-0.96(m, 6H), 0.58(m, 1H).

### EXAMPLE 1B

### (cis) (3R,5S)—N-(2-amino-2-oxoethyl)-3,5-dimethylcyclohexanecarboxamide

The product from Example 1A (300 g, 1.92 mol) and 1,1′-carbonyldiimidazole (CDI) (311 g, 1.92 mol) were combined in DMF (1.4 L) and stirred at room temperature for 1 hour. The mixture was treated with H₂O (10 mL), then treated with 2-aminoacetamide hydrochloride (234 g, 2.11 mol, purchased from Aldrich), and heated at 50° C. for 2 hours. The mixture was allowed to cool to room temperature and was treated with additional H₂O (8.4 L). The resulting precipitate was collected by filtration (H₂O wash) and dried. The solid was slurried in 3:1 H₂O/isopropanol, filtered (H₂O wash), and dried to afford the title compound as a solid. ¹H NMR (DMSO-d₆, 300 MHz) δ 8.12(m, 1H), 7.48(s, 1H), 7.20(s, 1H), 3.89-3.94(m, 2H), 246(m, 1H), 1.90(m, 2H), 1.81(m, 1H), 1.54-1.69(m, 2H), 1.05-1.17(m, 8H), 0.73(m, 1H); ¹³C NMR (DMSO-d₆, 100 MHz) δ 175.2, 171.2, 43.6, 43.1, 41.7, 37.2, 31.4, 22.5; MS m/z 213 (M+H)⁺.

### EXAMPLE 2

### 2-(3-methylcyclohexyl)acetamide

### EXAMPLE 2A

### ethyl(3-methylcyclohexylidene)acetate

Sodium hydride (2.65 g, 95% potency, 1.05 eq) in toluene (50 mL) was treated with triethyl phosphonoacetate (22.5 g, 100 mmol, purchased from Aldrich) dropwise keeping the temperature between 35-45° C. After complete addition and stirring for 1 hour at room temperature, the mixture was treated with 3-methylcyclohexanone (11.2 g, 100 mmol, purchased from Acros) and heated at 75-80° C. for 2 hours. After cooling to room temperature, the reaction mixture was treated with methanol:water (1:2, 150 mL) and then extracted with heptane (3×100 mL). The organic extracts were combined, washed with water (2×100 mL), and concentrated to provide the title compound as a 3:2 mixture of trans:cis isomers, which was used in the next step without further purification. ¹H NMR (CDCl₃, 300 MHz,) δ 1.05-1.20 (3H, d), 1.25 (3H, m), 1.40-2.35 (8H, m), 2.85-3.10 (1H, m), 4.18 (2H,m), 5.48 (1H, m).

### EXAMPLE 2B

### ethyl(3-methylcyclohexyl)acetate

The product from Example 2A (17.3 g, 0.104 mole) and 5% Pd/C (0.85 g) were combined in ethanol (20 mL) and treated with 40 psi of hydrogen for 2 hours. The mixture was filtered using ethanol (20 mL). The filtrate containing the title compound in ethanol was used in the next step.

### EXAMPLE 2C

### (3-methylcyclohexyl)acetic acid

The ethanolic solution from Example 2B was treated with 20 g of 50% aqueous NaOH at room temperature and stirred for 15 hours. The reaction mixture was diluted with water (50 mL) and extracted with heptane (50 mL). The aqueous phase was cooled to 0° C., acidified with aqueous HCl to pH 1-2, and extracted with heptane (3×100 mL). The heptane extracts were combined, washed with water, washed with brine, dried over MgSO₄, filtered, and the filtrate was concentrated under reduced pressure to provide the title compound. ¹H NMR (CDCl₃, 300 MHz) δ 0.65 (1H, q), 0.80-0.95 (4H, m), 1.20-1.80 (8H, m), 2.20-2.30 (2H, m), 11.30 (1H, s); MS m/z 157 (M+H)⁺.

### EXAMPLE 2D

### 2-(3-methylcyclohexyl)acetamide

The product from Example 2C (5.00 g, 32.1 mmol) and CDI (5.44 g, 33.6 mmol) were combined in ethyl acetate (50 mL) and stirred for 2 hours at room temperature. The mixture was then treated with 29% ammonium hydroxide (6 mL) and heated at 40-45° C. for 2 hours. The solution was diluted with ethyl acetate (50 mL) and washed sequentially with water (50 mL), citric acid (3×50 mL), water, and saturated aqueous Na₂CO₃. The organic extract was dried over MgSO₄, filtered, and the filtrate concentrated under reduced pressure to give a solid that was crystallized from ethyl acetate/heptane. The solid was collected by filtration and dried at 40-45° C. for 15 hours to provide the title compound. ¹H NMR (CDCl₃, 300 MHz) δ 0.60 (1H, q), 0.80-1.0 (4H, m), 1.08-1.80 (8H, m), 2.10 (2H, m), 5.60 (1H,s), 6.10 (1H, s); MS m/z 156 (M+H)⁺.

### EXAMPLE 3

### N-(2-amino-2-oxoethyl)-2-(3-methylcyclohexyl)acetamide

(3-Methylcyclohexyl)acetic acid (5.00 g, 32.1 mmol) and CDI (5.44 g, 33.6) were combined in ethyl acetate (50 mL) and stirred for 2 hours at room temperature. The solution was treated with 2-aminoacetamide hydrochloride (3.72 g, 33.7 mmol) and heated at 78-80° C. for 8 hours. The solution was diluted with ethyl acetate (50 mL), washed in succession with water (50 mL), citric acid (3×50 mL), saturated aqueous Na₂CO₃, dried over MgSO₄, filtered, and the filtrate was concentrated under reduced pressure. The residue was crystallized with ethyl acetate/heptane. The crystals were collected by filtration and dried at 40-45° C. for 15 hours to provide the title compound (3.3 g, 49% yield). ¹H NMR (DMSO-d₆, 300 MHz) δ 0.50 (1H, q), 0.65-0.85 (4H, m), 1.05-1.40 (4H, m), 1.50-2.0 (3H, m), 3.30 (3H, s), 3.60 (2H, s), 6.90 (1H, s), 7.20 (1H, s), 7.90 (1H, m); MS m/z 213 (M+H)⁺.

### EXAMPLE 4

### N-(2-amino-2-oxoethyl)-4-methylcyclohexanecarboxamide

4-Methylcyclohexanecarboxylic acid, purchased from Aldrich, was processed as described in Example 3 to provide the title compound. MS m/z 199 (M+H)⁺.

### EXAMPLE 5

### N-(2-amino-2-oxoethyl)-3-methylcyclohexanecarboxamide

3-Methylcyclohexanecarboxylic acid, purchased from Aldrich, was processed as described in Example 3 to provide the title compound. ¹H NMR (CDCl₃, 300 MHz) δ 6.3 (br s, 1H), 6.22 (br s, 1H), 5.46 (br s, 1H), 3.96 (d, 2H), 2.18 (tt, 1H), 0.90 (d, 3H), 1.15-1.85 (m, 9H); MS m/z 199 (M+H)⁺.

### EXAMPLE 6

### (1 S,2R,5S)—N-(2-amino-2-oxoethyl)-2,5-dimethylcyclohexanecarboxamide

### EXAMPLE 6A

### (1S,2R,5S)-2,5-dimethylcyclohexanecarboxylic acid

2,5-Dimethylbenzoic acid, purchased from Aldrich, was processed as described in Example 1 A to provide the title compound. The structure was determined by X-ray crystallography of its derivative of(1R, 2R)-(−)-pseudoephedrine. ¹H NMR (CDCl₃, 300 MHz) δ6.71 (sb, 1H), 6.56 (t, 1H, NH), 5.75 (sb, 1H), 4.00 (d, 2H), 2.43 (m), 2.27(m), 1.60(m), 1.44(m), 1.37 (m), 1.10(m), 0.94 (d, 3H), 0.86 (d, 3H); ¹³C NMR (CDCl₃, 100 MHz) δ 175.8, 171.5, 47.4, 42.8, 32.6, 32.4, 30.4, 30.4, 28.5, 22.6, 13.1; MS m/z 213 (M+H)⁺.

### EXAMPLE 6B

### (1 S,2R,5 S)—N-(2-amino-2-oxoethyl)-2,5-dimethylcyclohexanecarboxamide

The product from Example 6A was processed as described in Example 3 to provide the title compound. ¹H NMR (CDCl₃, 300 MHz) δ 6.71 (sb, 1H), 6.56 (t, 1H, NH), 5.75 (sb, 1H), 4.00 (d, 2H), 2.43 (m), 2.27(m), 1.60(m), 1.44(m), 1.37 (m), 1.10(m), 0.94 (d, 3H), 0.86 (d, 3H); ¹³C NMR (CDCl₃, 100 MHz) δ 175.8, 171.5, 47.4, 42.8, 32.6, 32.4, 30.4, 30.4, 28.5, 22.6, 13.1; MS m/z 213 (M+H)⁺.

### EXAMPLE 7

### N-(2-amino-2-oxoethyl)-2,3-dimethylcyclohexanecarboxamide

### EXAMPLE 7A

### 2,3-dimethylcyclohexanecarboxylic acid

2,3-Dimethylbenzoic acid, purchased from Aldrich, was processed as described in Example 1A to provide the title compound as a mixture of diastereomers.

### EXAMPLE 7B

### N-(2-amino-2-oxoethyl)-2,3-dimethylcyclohexanecarboxamide

The product from Example 7A was processed as described in Example 3 to provide the title compound. ¹H NMR (CDCl₃, 300 MHz) δ 6.58 (brs, 1H), 6.46 (br s, 1H), 5.64 (br s, 1H), 3.96 (2H,), 2.38 (tt, 1H), 2.12 (m, 1H), 1.76 (m, 1H), 1.18-1.68 (m, 7H), 0.88 (d, 3H), 0.72 (d, 3H); ¹³C NMR (CDCl₃, 100 MHz) δ 176.1, 171.9, 49.0, 42.6, 36.6, 36.1, 27.6, 25.4, 21.2, 19.9, 7.1; MS m/z 213 (M+H)⁺.

### EXAMPLE 8

### N-(2-amino-2-oxoethyl)-2-methylcyclohexanecarboxamide

### EXAMPLE 8A

### 2-methylcyclohexanecarboxylic acid

2-Methylbenzoic acid, purchased from Aldrich, was processed as described in Example 1A to provide the title compound.

### EXAMPLE 8B

### N-(2-amino-2-oxoethyl)-2-methylcyclohexanecarboxamide

The product from Example 8A was processed as described in Example 3 to provide the title compound. ¹H NMR (CD₃OD, 300 MHz) δ 3.8 (2H), 2.45 (1H), 2.14 (1H), 1.2-1.8 (m, 8H), 0.9 (d, 3H); MS m/z 199 (M+H)⁺.

### EXAMPLE 9

### N-(2-amino-2-oxoethyl)-3,4-dimethylcyclohexanecarboxamide

### EXAMPLE 9A

### 3,4-dimethylcyclohexanecarboxylic acid

3,4-Dimethylbenzoic acid, purchased from Aldrich, was processed as described in Example 1A to provide the title compound as a mixture of diastereomers. ¹H NMR (CDCl₃, 300 MHz) δ 11.8 (br s, 1H), 2.34 (m, 1H), 0.94 (d, 3H), 0.88 (d, 3H), 1.3-1.8 (m, 8H); ¹³C NMR (CDCl₃, 100 MHz) δ 183.0,43.5,34.2, 32.4, 32.0,30.5,22.4, 19.7, 11.4; MS m/z 174 (M+NH₄)⁺.

### EXAMPLE 9B

### N-(2-amino-2-oxoethyl)-3,4-dimethylcyclohexanecarboxamide

The product from Example 9A was processed as described in Example 3 to provide the title compound. ¹H NMR (CD₃OD, 300 MHz) δ 11.8 (br s, 1H), 2.34 (m, 1H), 0.94 (d, 3H), 0.88 (d, 3H), 1.3-1.8 (m, 8H); ¹³C NMR (CD₃OD, 100 MHz) δ 183.0, 43.5, 34.2, 32.4, 32.0, 30.5, 22.4, 19.7, 11.4; MS m/z 174 (M+NH₄)⁺.

### EXAMPLE 10

### N-(2-amino-2-oxoethyl)-4-isopropylcyclohexanecarboxamide

### EXAMPLE 10A

### 4-isopropylcyclohexanecarboxylic acid

4-Isopropylbenzoic acid, purchased from Aldrich, was processed as described in Example 1A to provide the title compound as a mixture of diastereomers. MS m/z 188 (M+NH₄)⁺.

### EXAMPLE 10B

### N-(2-amino-2-oxoethyl)-4-isopropylcyclohexanecarboxamide

The product from Example 10A was processed as described in Example 3 to provide the title compound. MS m/z 227 (M+H)⁺.

### EXAMPLE 11

### N-(2-amino-2-oxoethyl)-4-tert-butylcyclohexanecarboxamide

### EXAMPLE 11A

### 4-tert-butylcyclohexanecarboxylic acid

4-tert-Butylbenzoic acid, purchased from Aldrich, was processed as described in Example 1A to provide the title compound as mixture of diastereomers.

### EXAMPLE 11B

### N-(2-amino-2-oxoethyl)-4-tert-butylcyclohexanecarboxamide

The product from Example 11A was processed as described in Example 3 to provide the title compound. MS m/z 241 (M+H)⁺.

### EXAMPLE 12

### N-(2-amino-2-oxoethyl)bicyclo[2.2.1]heptane-2-carboxamide

Bicyclo[2.2.1]heptane-2-carboxylic acid (5.0 g, 25.7 mmol, purchased from Lancaster) and 5% Pd/C (0.5 g) were combined in methanol (200 mL) and treated with hydrogen gas at 123 psi with sonication for 30 minutes. The mixture was filtered and the filtrate concentrated to provide a solid (5.34 g). The solid was slurried in ethyl acetate (30 mL) overnight at room temperature and then filtered to provide the title compound (4.55 g, 90% yield). ¹H NMR (CD₃OD, 300 MHz) δ 3.92 (d, 2H), 3.76 (d,2H), 2.78 (m, 1H), 2.52 (m, 1H), 2.22 (m, 1H), 1.20-1.62 (m, 8H); MS m/z 197 (M+H)⁺.

### EXAMPLE 13

### N-(2-amino-2-oxoethyl)-1-adamantanecarboxamide

1-Adamantanecarboxylic acid, purchased from Aldrich, was processed as described in Example 3 to provide the title compound. ¹H NMR (CD₃OD, 300 MHz) δ 3.80 (s, 2H), 2.04 (m, 3H), 1.88 (m, 6H), 1.76 (m, 6H); MS m/z 237 (M+H)⁺.

### EXAMPLE 14

### N-(2-amino-2-oxoethyl)hexahydro-2,5-methanopentalene-3a(1H)-carboxamide

Hexahydro-2,5-methanopentalene-3a(1H)-carboxylic acid, purchased from Acros, was processed as described in Example 3 to provide the title compound. ¹H NMR (CD₃OD, 300 MHz) δ 3.94 (s), 2.72 (t, 1H), 2.32 (m, 2H), 2.04 (m, 2H), 1.84 (m, 4H), 1.66 (m, 4H); ¹³C NMR (CD₃OD, 100 MHz) δ 180.8, 174.5, 56.3, 48.3, 44.7, 43.3, 39.0, 35.8; MS m/z 223 (M+H)⁺.

### 424423 EXAMPLE 15

### N-(2-amino-2-oxoethyl)-2,4-dimethylcyclohexanecarboxamide

### EXAMPLE 15A

### 2,4-dimethylcyclohexanecarboxylic acid

2,4-Dimethylbenzoic acid, purchased from Aldrich, was processed as described in Example 1A to provide the title compound as a mixture of diastereomers. ¹H NMR (CDCl₃, 300 MHz) 11.0 (br s, 1H), 2.62 (dt, 1H), 2.04 (dq, 1H), 1.04 (d, 3H), 0.92 (d, 3H), 1.24-1.75 (m, 7H); MS m/z 174 (M+NH₄)⁺.

### EXAMPLE 15B

### N-(2-amino-2-oxoethyl)-2,4-dimethylcyclohexanecarboxamide

The product from Example 15A was processed as described in Example 3 to provide the title compound. ¹H NMR (CD₃OD, 300 MHz) δ 3.78 (2H), 2.66 (m, 1H), 1.94 (dq, 1H), 0.96 (s, 3H), 0.90 (d, 3H), 1.3-1.75 (m, 7H); MS m/z 213 (M+H)⁺.

### EXAMPLE 16

### (cis) (3R,5S)—N-[(1S)-2-amino-1-methyl-2-oxoethyl]-3,5-dimethylcyclohexanecarboxamide

The product from Example 1A was processed as described in Example 3 substituting (2S)-2-aminopropanamide hydrochloride, purchased from Aldrich, for 2-aminoacetamide hydrochloride to provide the title compound. ¹H NMR (DMSO-d₆, 300 MHz) δ 7.71(m, 1H), 7.21(s, 1H), 6.90(s, 1H), 4.17(m, 1H), 2.25(m, 1H), 1.55-1.70(m, 3H), 1.30-1.46(m, 2H), 1.13-1.20(m, 3H), 0.84-0.92(m, 8H), 0.51(m, 1H); ¹³C NMR (DMSO-d₆, 100 MHz) δ 174.6, 174.5, 47.6, 43.5, 43.1, 37.2, 37.1, 31.3, 31.3, 22.5, 22.5, 18.3; MS m/z 227 (M+H)⁺.

### EXAMPLE 17

### (cis) (3R,5S)—N-(3-amino-3-oxopropyl)-3,5-dimethylcyclohexanecarboxamide

The product from Example 1A was processed as described in Example 3 substituting 3-aminopropanamide hydrochloride, purchased from Chem-Impex Tools For Peptide and Solid Phase Synthesis, for 2-aminoacetamide hydrochloride to provide the title compound. ¹H NMR (CD₃OD, 300 MHz) δ 3.34 (t, 2H), 2.39 (t, 2H), 2.22 (tt, 1H), 1.72 (m, 2H), 1.65 (m, 1H), 1.5-1.4 (m, 2H), 0.99 (q, 2H), 0.91 (d, 6H), 0.55(q, 1H); ¹³C NMR (CD₃OD, 100 MHz): 178.4, 175.9, 46.3, 44.7, 38.8, 36.8, 36.1, 33.3, 23.0. MS m/z 227 (M+H)³⁰ .

### EXAMPLE 18

### (cis) (3R,5S)—N-[(1R)-2-amino-1-methyl-2-oxoethyl]-3,5-dimethylcyclohexanecarboxamide

The product from Example 1A was processed as described in Example 3 substituting (2R)-2-aminopropanamide hydrochloride, purchased from Bachem, for 2-aminoacetamide hydrochloride to provide the title compound. ¹H NMR (CD₃OD, 300 MHz) δ 4.31 (q, 1H), 2.31 (tt, 1H), 1.8-1.6 (m, 3H), 1.5-1.4 (m, 2H), 1.33 (d, 3H), 1.00 (q, 2H), 0.92 (d, 6H), 0.56(q, 1H); ¹³C NMR (CD₃OD, 100 MHz) δ 178.0, 177.2, 45.9, 44.7, 38.8, 38.5, 33.3, 33.2, 23.1 and 18.5. MS m/z 227 (M+H)⁺.

### EXAMPLE 19

### (cis) (3R,5S)—N-[(1S)-2-amino-1-(1H-imidazol-4-ylmethyl)-2-oxoethyl]-3,5-dimethylcyclohexanecarboxamide

The product from Example 1A was processed as described in Example 3 substituting (2S)-2-amino-3-(1H-imidazol-4-yl)propanamide dihydrochloride, purchased from Bachem, for 2-aminoacetamide hydrochloride to provide the title compound.

¹H NMR (DMSO-D₆, 400 MHz), 11.5 (br s, 1H), 7.76 (d, 1H), 7.48 (d, 1H), 7.19 (s, 1H), 6.94 (s, 1H), 6.74 (s, 1H), 4.36 (m, 1H), 2.86 (dd, 1H), 2.74 (dd, 1H), 2.19 (tt, 1H), 1.7-1.5 (m, 3H), 1.42-1.3 (m, 2H), 0.92 (m, 1H), 0.91 (d, 6H), 0.48 (q, 1H). MS m/z, 293 (M+H)⁺.

### EXAMPLE 20

### (cis) (3R,5S)—N-[(1S)-1-(aminocarbonyl)-2-methylpropyl]3,5-dimethylcyclohexanecarboxamide

The product from Example 1A was processed as described in Example 3 substituting (2S)-2-amino-3-methylbutanamide hydrochloride, purchased from Bachem, for 2-aminoacetamide hydrochloride to provide the title compound. ¹H NMR (CD₃OD, 400 MHz) δ 4.17 (d, 1H), 2.38 (tt, 1H), 2.04 (m, 1H), 1.8-1.6 (m, 3H), 1.5-1.4 (m, 2H), 1.1-0.91 (m, 14 H), 0.57 (m, 1H); ¹³C NMR (CD₃OD, 100 MHz) δ 178.2, 175.8, 59.4, 46.0, 44.7, 39.2, 38.4, 33.3, 33.2, 31.9, 23.1, 19.9, 18.7; MS m/z, 255 (M+H)⁺.

### EXAMPLE 21

### (cis) (2S)-2-({[(3R,5S)-3,5-dimethylcyclohexyl]carbonyl}amino)propanoic acid

The product from Example 1A was processed as described in Example 3 substituting (2S)-2-aminopropanoic acid for 2-aminoacetamide hydrochloride to provide the title compound. ¹H NMR (CDCl₃, 400 MHz) δ 8.30 (br, 1H), 6.26 (d, 1H) 4.58 (m, 1H), 2.25 (tt, 1H), 1.85 (m, 2H), 1.67 (m, 1H), 1.32 (m, 1H), 1.30 (d, 3H), 1.05 (m, 2H), 0.93 (d, 6H), 0.60 (m, 1H); MS m/z, 228 (M+H)⁺.

### EXAMPLE 22

### 2-cyclopentylacetamide

Cyclopentylacetic acid, purchased from Aldrich, was processed as described in Example 2D to provide the title compound. ¹H NMR (CDCl₃, 300 MHz) δ 1.20 (2H, m), 1.60 (4H, m), 1.85 (2H, m), 2.22 (3H, m), 5.50 (1H,s), 5.70 (1H, s); MS m/z 128 (M+H)⁺.

### EXAMPLE 23

### N-(2-amino-2-oxoethyl)-2-cyclopentylacetamide

Cyclopentylacetic acid was processed as described in Example 3 to provide the title compound. ¹H NMR (CDCl₃, 300 MHz) δ 1.10 (2H, m), 1.20-1.65 (6H, m), 2.10 (2H, s), 3.30 (1H, s), 3.60 (2H, d), 6.86 (1H, s), 7.20 (1H, s), 7.85 (1H, t); MS m/z 185 (M+H)⁺.

### EXAMPLE 24

### 2-cyclohexylacetamide

Cyclohexylacetic acid, purchased from Aldrich, was processed as described in Example 2D to provide the title compound. ¹H NMR (CDCl₃, 300 MHz) δ 0.90-1.40 (5H, m), 1.60-1.80 (6H, m), 2.10 (2H, d), 5.42 (1H, s), 5.70 (1H, s); MS m/z 142 (M+H)⁺.

### EXAMPLE 25

### N-(2-amino-2-oxoethyl)-2-cyclohexylacetamide

Cyclohexylacetic acid was processed as described in Example 3 to provide the title compound. ¹H NMR (DMSO-d₆, 300 MHz) δ 0.85 (2H, m), 1.16 (3H, m), 1.60 (5H, m), 1.98 (2H, d), 3.30 (1H, s), 3.60 (2H, d), 6.95 (1H, s), 7.20 (1H, d), 7.85 (1H, t); MS m/z 199 (M+H)⁺.

### EXAMPLE 26

### 2-(4-methylcyclohexyl)acetamide

(4-Methylcyclohexyl)acetic acid, purchased from Aldrich, was processed as described in Example 2D to provide the title compound. MS m/z 156 (M+H)⁺.

### EXAMPLE 27

### N-(2-amino-2-oxoethyl)-2-(4-methylcyclohexyl)acetamide

(4-Methylcyclohexyl)acetic acid was processed as described in Example 3 to provide the title compound. ¹H NMR (DMSO-d₆, 300 MHz) δ 0.85 (5H, m), 1.20-1.60 (6H, m), 1.98 (3H, d), 3.30 (1H, s), 3.60 (2H, d), 6.95 (1H, s), 7.25 (1H, s), 7.90 (1H, t); MS m/z 213 (M+H)⁺.

### EXAMPLE 28

### 2-(2-methylcyclohexyl)acetamide

### EXAMPLE 28A

### (2-methylcyclohexyl)acetic acid

(2-Methylphenyl)acetic acid was processed as described in Example 1A to provide the title compound. ¹H NMR (CDCl₃, 300 MHz) δ 0.85 (3H, q), 1.0-1.85 (7H, m), 2.05 (2H, m), 2.25 (2H, m), 2.60 (1H, q), 11.25 (1H, s); MS m/z 157 (M+H)⁺.

### EXAMPLE 28B

### 2-(2-methylcyclohexyl)acetamide

The product from Example 28A was processed as described in Example 2D to provide the title compound. ¹H NMR (DMSO-d₆, 300 MHz) δ 1.05 (3H, q), 1.06-1.65 (5H, m), 1.80-2.20 (5H, m), 3.28 (2H, s), 6.90 (1H, s), 7.45 (1H, s); MS m/z 156 (M+H)⁺.

### EXAMPLE 29

### N-(2-amino-2-oxoethyl)-2-(2-methylcyclohexyl)acetamide

(2-Methylcyclohexyl)acetic acid was processed as described in Example 3 to provide the title compound. ¹H NMR (DMSO-d₆, 300 MHz) δ 0.98 (3H, q), 1.05-2.10 (8H, m), 3.45 (4H, s), 3.72 (2H, d), 7.15 (1H, s), 7.40 (1H, s), 8.10 (1H, m); MS m/z 213 (M+H)⁺.

### EXAMPLE 31

### 2-(5-isopropyl-2-methylcyclohexyl)acetamide

### EXAMPLE 31A

### ethyl (5-isopropenyl-2-methylcyclohexylidene)acetate

(+)-Dihydrocarvone, purchased from Aldrich, was processed as described in Examples 2A to provide the title compound.

### EXAMPLE 31B

### ethyl (5-isopropyl-2-methylcyclohexyl)acetate

Ethyl (5-isopropenyl-2-methylcyclohexylidene)acetate was processed as described in Example 2B to provide the title compound.

### EXAMPLE 31C

### (5-isopropyl-2-methylcyclohexyl)acetic acid

Ethyl (5-isopropyl-2-methylcyclohexyl) acetate was processed as described in Example 2C to provide the title compound. ¹H NMR (CDCl₃, 300 MHz) δ 0.80-1.0 (10H, m), 1.05-2.05 (8H, m), 2.20-2.65 (3H, m); MS m/z 199 (M+H)⁺.

### EXAMPLE 31D

### 2-(5-isopropyl-2-methylcyclohexyl)acetamide

(5-Isopropyl-2-methylcyclohexyl)acetic acid was processed as described in Example 2D to provide the title compound. ¹H NMR (CDCl₃, 300 MHz) δ 0.80-1.0 (10H, m), 1.05-1.90 (8H, m), 2.0-2.45 (3H, m), 5.70 (1H, s), 6.15 (1H, s); MS m/z 198 (M+H)⁺.

### EXAMPLE 32

### N-(2-amino-2-oxoethyl)-2-(5-isopropyl-2-methylcyclohexyl)acetamide

(5-Isopropyl-2-methylcyclohexyl)acetic acid was processed as described in Example 3 to provide the title compound. ¹H NMR (CDCl₃, 300 MHz) δ 0.80-1.10 (10H, m), 1.20-2.45 (11H, m), 3.98 (2H, m), 6.0 (1H, s), 6.85 (2H, m); MS m/z 255 (M+H)⁺.

### EXAMPLE 33

### 2-(4-tert-butylcyclohexyl)acetamide

### EXAMPLE 33A

### ethyl (4-tert-butylcyclohexylidene)acetate

4-tert-Butylcyclohexanone, purchased from Aldrich, was processed as described in Example 2A to provide the tile compound.

### EXAMPLE 33B

### ethyl (4-tert-butylcyclohexyl)acetate

The product from Example 33A was processed as described in Example 2B to provide the tile compound.

### EXAMPLE 33C

### (4-tert-butylcyclohexyl)acetic acid

The product from Example 33B was processed as described in Example 2C to provide the tile compound. ¹H NMR (CDCl₃, 300 MHz) δ 0.80 (9H, m), 1.0-1.20 (2H, m), 1.45-1.90 (8H, m), 2.20-2.40 (2H, m); MS m/z 216 (M+NH₄)⁺.

### EXAMPLE 33D

### 2-(4-tert-butylcyclohexyl)acetamide

(4-tert-Butylcyclohexyl)acetic acid was processed as described in Example 2D to provide the tile compound. ¹H NMR (CDCl₃, 300 MHz) δ 0.80-1.0 (10H, m), 1.05 (1H, m), 1.15-2.05 (10H, m), 6.65 (1H, s), 7.20 (1H, dd); MS m/z 198 (M+H)⁺.

### EXAMPLE 34

### N-(2-amino-2-oxoethyl)-2-(4-tert-butylcyclohexyl)acetamide

(4-tert-Butylcyclohexyl)acetic acid was processed as described in Example 3 to provide the tile compound. ¹H NMR (DMSO-d₆, 300 MHz) δ 0.80-1.0 (10H, m), 1.10 (1H, m), 1.25-1.80 (5H, m), 1.98 (1H, d), 2.15 (2H, m), 3.30 (2H, s), 3.60 (2H, m), 6.95 (1H, s), 7.20 (1H, s), 7.85-8.0 (1H, m); MS m/z 225 (M+H)⁺.

### EXAMPLE 35

### 2-bicyclo[2.2.1]hept-2-ylacetamide

Bicyclo[2.2.1]hept-2-ylacetic acid, purchased from Aldrich, was processed as described in Example 2D to provide the title compound as a mixtue of endo and exo isomers. ¹H NMR (CDCl₃, 300 MHz) δ 1.0-1.30 (5H, m), 1.40-1.60 (3H, m), 1.85 (1H, m), 2.0-2.25 (4H, m), 5.45 (1H, s), 5.70 (1H, s); MS m/z 154 (M+H)⁺.

### EXAMPLE 36

### N-(2-amino-2-oxoethyl)-2-bicyclo[2.2.1]hept-2-ylacetamide

The title compound can be prepared by processing bicyclo[2.2.1]hept-2-ylacetic acid as described in Example 3.

### EXAMPLE 37

### 2-(1-adamantyl)acetamide

1-Adamantylacetic acid, purchased from Aldrich, was processed as described in Example 2D to provide the tile compound. ¹H NMR (CDCl₃, 300 MHz) δ 1.60-1.80 (10H, m), 1.90-2.10 (7H, m), 5.30 (1H, s), 5.60 (1H, s); MS m/z 194 (M+H)⁺.

### EXAMPLE 38

### 2-(1-adamantyl)-N-(2-amino-2-oxoethyl)acetamide

1-Adamantylacetic acid was processed as described in Example 3 to provide the tile compound. ¹H NMR (DMSO-d₆, 300 MHz) δ 1.45-1.62 (9H, m), 1.90 (4H, s), 3.36 (4H, s), 3.60 (2H, d), 6.90 (1H, s), 7.20 (1H, s), 7.80 (1H, t); MS m/z 251 (M+H)⁺.

### EXAMPLE 39

### 2-(4,4-dimethylcyclohexyl)acetamide

### EXAMPLE 39A

### ethyl (4,4-dimethyl-cyclohex-2-enylidene)acetate

4,4-Dimethyl-2-cyclohexene-1-one, purchased from Aldrich, was processed as described in Example 2A to provide the title compund.

### EXAMPLE 39B

### ethyl (4,4-dimethylcyclohexyl)acetate

Ethyl (4,4-dimethyl-cyclohex-2-enylidene)acetate was processed as described in Example 2B to provide the title compund.

### EXAMPLE 39C

### (4,4-dimethylcyclohexyl)acetic acid

Ethyl (4,4-dimethylcyclohexyl)acetate was processed as described in Example 2C to provide the title compund. ¹H NMR (CDCl₃, 300 MHz) δ 0.85-1.0 (6H, m), 1.05-1.40 (6H, m), 1.45-2.60 (5H, m), 11.45 (1H, s); MS m/z 171 (M+H)⁺.

### EXAMPLE 39D

### 2-(4,4-dimethylcyclohexyl)acetamide

The title compound can be preparded by processing (4,4-dimethylcyclohexyl)acetic acid as described in Example 2D.

### EXAMPLE 40

### N-(2-amino-2-oxoethyl)-2-(4,4-dimethylcyclohexyl)acetamide

The title compound can be preparded by processing (4,4-dimethylcyclohexyl)acetic acid as described in Example 3.

### EXAMPLE 41

### 1,1′:1′,1″-ter(cyclopropane)-2′-carboxamide

### EXAMPLE 41A

### ethyl 1,1′:1′,1″-ter(cyclopropane)-2′-carboxylate

(1-Cyclopropylvinyl)cyclopropane (1.0 equivalents, commercially available from Alfa-DM) and copper powder (about 15% by wt) were combined in methyl cyclohexane (approximately 4M) and heated to 105° C. Ethyl diazoacetate (1.1 equivalents, commercially available from Aldrich) was added dropwise over an 8-hour period while maintaining the temperature between 100-105° C. Upon complete addition, the mixture was heated an additional 2 hours, allowed to cool to ambient temperature and stirred for an additional 12 hours. The reaction mixture was filtered and concentrated under reduced pressure to afford the title compound as a mixture of diastereomers which was used without further purification.

### EXAMPLE 41B

### 1,1′:1′,1″-ter(cyclopropane)-2′-carboxylic acid

The product from Example 41A in water (about 2M) was treated with NaOH (about 1.5 equivalents) and heated at reflux for about 8 hours. The reaction mixture was allowed to cool to ambient temperature and extracted with diethyl ether. The aqueous layer was acidified to pH 3 by careful addition of concentrated HCl and extracted with diethyl ether (3×100 mL). The ethereal extracts were combined and concentrated under reduced pressure to afford the title compound. MS (ESI) m/z 165 (M−H)⁻.

### EXAMPLE 41C

### 1,1′:1′,1″-ter(cyclopropane)-2′-carboxamide

1,1′:1′,1″-Ter(cyclopropane)-2′-carboxylic acid was processed as described in Example 2D to provide the title compound. MS (APCI) m/z 166 (M+H)⁺.

### EXAMPLE 42

### N-(2-amino-2-oxoethyl)-1,1′:1′,1″-ter(cyclopropane)-2′-carboxamide

1,1′:1′,1″-Ter(cyclopropane)-2′-carboxylic acid was processed as described in Example 3 to provide the title compound. MS (APCI) m/z 223 (M+H)⁺.

### EXAMPLE 43

### (cis) (3R,5S)-3,5-dimethylcyclohexanecarboxamide

(cis) (3R,5S)-3,5-Dimethylcyclohexanecarboxylic acid was processed as described in Example 2D to provide the title compound.

### EXAMPLE 44

### 2,3-dimethylcyclohexanecarboxamide

2,3-Dimethylcyclohexanecarboxylic acid can be processed as described in Example 2D to provide the title compound.

### EXAMPLE 45

### 4-methylcyclohexanecarboxamide

4-Methylcyclohexanecarboxylic acid can be processed as described in Example 2D to provide the title compound.

### EXAMPLE 46

### 3-methylcyclohexanecarboxamide

3-Methylcyclohexanecarboxylic acid can be processed as described in Example 2D to provide the title compound.

### EXAMPLE 47

### 2-methylcyclohexanecarboxamide

2-Methylcyclohexanecarboxylic acid can be processed as described in Example 2D to provide the title compound.

### EXAMPLE 48

### cyclopentanecarboxamide

Cyclopentanecarboxylic acid can be processed as described in Example 2D to provide the title compound.

### EXAMPLE 49

### 2,5-dimethylcyclohexanecarboxamide

### EXAMPLE 49A

### 2,5-dimethylcyclohexanecarboxylic acid

The title compound was prepared as a mixture of diastereomers according to the procedure described in Smith, H. A. ; Stanfield, J. A. , J. Amer. Chem. Soc. 1949, 71, 81-83. ¹H NMR (CDCl₃, 300 MHz) δ 2.58 (dt, 1H), 2.38 (m, 1H), 1.68 (m, 1H), 1.59 (m, 2H), 1.44 (m, 1H), 1.3-1.4 (m, 1H), 1.3 (m, 1H), 1.0-1.2 (m, 1H), 0.94 (d, 3H), 0.92 (d, 3H); MS m/z 174 (M+NH₄)⁺.

### EXAMPLE 49B

### 2,5-dimethylcyclohexanecarboxamide

2,5-Dimethylcyclohexanecarboxylic acid can be processed as described in Example 2D to provide the title compound.

### EXAMPLE 50

### 3,4-dimethylcyclohexanecarboxamide

3,4-Dimethylcyclohexanecarboxylic acid can be processed as described in Example 2D to provide the title compound.

### EXAMPLE 51

### 4-isopropylcyclohexanecarboxamide

4-Isopropylcyclohexanecarboxylic acid can be processed as described in Example 2D to provide the title compound.

### EXAMPLE 52

### 4-tert-bautylcyclohexanecarboxamide

4-tert-Butylcyclohexanecarboxylic acid can be processed as described in Example 2D to provide the title compound.

### EXAMPLE 53

### bicyclo[2.2.1]heptane-2-carboxamide

Bicyclo[2.2.1]heptane-2-carboxylic acid can be processed as described in Example 2D to provide the title compound.

### EXAMPLE 54

### 1-adamantanecarboxamide

1-Adamantanecarboxylic acid can be processed as described in Example 2D to provide the title compound.

### EXAMPLE 55

### hexahydro-2,5-methanopentalene-3a(1H)-carboxamide

Hexahydro-2,5-methanopentalene-3a(1H)-carboxylic acid can be processed as described in Example 2D to provide the title compound.

### EXAMPLE 56

### 2,4-dimethylcyclohexanecarboxamide

2,4-Dimethylcyclohexanecarboxylic acid can be processed as described in Example 2D to provide the title compound.

### EXAMPLE 57

### (cis) (2R,6S)-2,4,6-trimethylcyclohexanecarboxamide

### EXAMPLE 57A

### (cis) (2R,6S)-2,4,6-trimethylcyclohexanecarboxylic acid

2,4,6-Trimethylbenzoic acid, purchased from Aldrich, was processed as described in Example 1A to provide the title compound. ¹H NMR (CDCl₃, 300 MHz) 11.2 (br s, 1H), 2.56 (t, 1H), 1.76 (m, 2H), 1.2-1.4 (m), 0.98 (d, 6H), 0.92 (d, 3H); MS m/z 188 (M+NH₄)⁺.

### EXAMPLE 57B

### (cis) (2R,6S)-2,4,6-trimethylcyclohexanecarboxamide

(cis) (2R,6S)-2,4,6-trimethylcyclohexanecarboxylic acid can be processed as described in Example 2D to provide the title compound.

### EXAMPLE 58

### N-(2-amino-2-oxoethyl)cyclopentanecarboxamide

Cyclopentanecarboxylic acid can be processed as described in Example 3 to provide the title compound.

### EXAMPLE 59

### N-(2-amino-2-oxoethyl)-2,5-dimethylcyclohexanecarboxamide

2,5-Dimethylcyclohexanecarboxylic acid can be processed as described in Example 3 to provide the title compound.

### EXAMPLE 60

### (cis) (2R,6S)—N-(2-amino-2-oxoethyl)-2,4,6-trimethylcyclohexanecarboxamide

(cis) (2R,6S)-2,4,6-trimethylcyclohexanecarboxylic acid can be processed as described in Example 3 to provide the title compound.

### EXAMPLE 61

### (cis) (3R,5S)—N-[(2R)-2-hydroxypropyl]-3,5-dimethylcyclohexanecarboxamide

(cis) (3R,5S)-3,5-Dimethylcyclohexanecarboxylic acid was processed as described in Example 3 substituting (2R)-1-amino-2-propanol, purchased from Aldrich, for 2-aminoacetamide hydrochloride to provide the title compound. ¹H NMR (400 MHz, CD₃OD) δ 3.80 (m, 1H), 3.11 (m, 2H), 2.26 (tt, 1H), 1.40-1.70 (m, 5H), 1.12 (d, 3H), 1.00 (m, 2H), 0.92 (d, 6H), 0.57 (m, 1H); ¹³C NMR (100 MHz, CD₃OD) δ 178.6, 67.4, 47.6, 46.3, 44.7, 38.8, 33.3, 23.1, 21.1.

### EXAMPLE 62

### (cis) (3R,5S)—N-[(2S)-2-hydroxypropyl]-3,5-dimethylcyclohexanecarboxamide

(cis) (3R,5S)-3,5-Dimethylcyclohexanecarboxylic acid was processed as described in Example 3 substituting (2S)-1-amino-2-propanol, purchased from Aldrich, for 2-aminoacetamide hydrochloride to provide the title compound. ¹H NMR (400 MHz, CD₃OD) δ 3.80 (m, 1H), 3.11 (m, 2H), 2.26 (tt, 1H), 1.40-1.70 (m, 5H), 1.12 (d, 3H), 1.00 (m, 2H), 0.92 (d, 6H), 0.57 (m, 1H); ¹³C NMR (100 MHz, CD₃OD) δ 178.6, 67.4, 47.6, 46.3, 44.7, 38.8, 33.3, 23.1, 21.1

### EXAMPLE 63

### (3R,5S)—N,3,5-trimethylcyclohexanecarboxamide

The title compound can be prepared using the procedure described in Example 1B except substituting N-methylamine for 2-aminoacetamide hydrochloride.

### Determination of Anticonvulsant Effect

The anticonvulsant effect of a representative number of compounds of the present invention were determined using the procedures described hereinafter.

Adult, male, CD-1 mice (22-25 grams) were obtained from Charles River Laboratories (Portage, Mich.) and housed at Abbott Laboratories (Abbott Park) under standard lighting conditions of 12 hours on/12 hours off, with lights on at 6 a.m. Food and water were provided ad libitum and mice weighed 25-35 grams at the time of testing.

Compounds were prepared for oral administration by suspending them in a vehicle of 100 μL Tween® 80 per mL hydroxypropyl methylcellulose (2 mg/mL, Abbott Laboratories). Compound solutions were administered at a volume of 10 mL/kg, p.o.

Maximal Electroshock Procedure

The method used was similar to that of E. A. Swinyard, General principles: Experimental selection, quantification and evaluation of anti-convulsants, Anti-epileptic Drugs, Third Edition, R. Levey, et al., Editors. 1989, Raven Press Ltd: New York. Mice were pretreated orally with compounds of the present invention 30 minutes prior to electrical stimulation. Electrical stimulation consisted of pulsed electrical current (50 mA, 0.4 second duration, pulse width 0.5 msec, 60 pulses/sec) applied via corneal electrodes to induce seizure. The stimulation was delivered with an ECT Unit (Ugo Basile #7801). The electrodes of the unit were coated with electrocardiogram electrolyte (Signa Creme, Parker Labs #1708) to insure good contact with the corneas. Mice were observed post-stimulation for the onset of tonic seizures and death. Mice were considered to have had a tonic seizure only if there was a prolonged extension (>90° from plane of body) of the hind legs. Mice were assigned scores of either “positive” or “negative.” A positive score indicated that the symptom was present; a negative that it was not. Those that did not seize were considered protected. A total of 20 mice were used in each group. The percent protection from tonic seizures was calculated by dividing the number of protected mice by the total number in the group. The ED₅₀ for the compounds were calculated using PROBIT analysis and represent the dose at which 50% of the mice were protected from tonic seizures. Valproate exhibited an ED₅₀ of 1.2 mmol/kg. Representative compounds of the present invention exhibited ED₅₀ₛ in the range of about 0.70 mmol/kg to about 0.13 mmol/kg.

Subcutaneous Pentylenetetrazole (PTZ) Seizure Procedure

The method used was similar to that of E. A. Swinyard, General principles: Experimental selection, quantification and evaluation of anti-convulsants, Anti-epileptic Drugs, Third Edition, R. Levey, et al., Editors. 1989, Raven Press Ltd: New York. During the experiment the mice were housed individually in clear polycarbonate cages for observation. Mice, excluding control, were pretreated orally with a compound of the present invention 30 minutes prior to PTZ injection and were observed for 15 minutes following administration of PTZ. Seizures were induced by the subcutaneous injection of pentylenetetrazole (PTZ, 85 mg/kg) just below the nape of the neck. Time to clonic and tonic seizures was noted, and the number of mice that exhibited seizures was recorded. A total of 20 mice were used in each group. The ED₅₀ for the compounds were calculated using linear regression and represent the dose at which 50% of the mice were protected from tonic seizures. Valproate exhibited an ED₅₀ of 1.8 mmol/kg. Representative compounds of the present invention exhibited ED₅₀ₛ in the range of about 1.0 mmol/kg to about 0.60 mmol/kg.

Compounds of the present invention can be used to treat seizures including, but not limited to, epilepsy as described by Schmidt, D., The clinical impact of new antiepileptic drugs after a decade of use in epilepsy, Epilepsy Res., 2002, 50(1-2), 21-32; Asconape, J. J., Some common issues in the use of antiepileptic drugs, Seminars in Neurology, 2002, 22(1), 27-39; and Wallace, S. J., Newer antiepileptic drugs: advantages and disadvantages, Brain & Development, 2001, 23, 277-283.

Compounds of the present invention can be used to treat bipolar disorder as described by Brambilla, P., Barale, F., Soares, J. C., Perspectives on the use of anticonvulsants in the treatment of bipolar disorder, International Journal of Neuropsychopharmacology, 2001, 4, 421-446; Angel, I. and Horovitz, T., Bipolar disorder and valproic acid, Current Opinion in Central & Peripheral Nervous System Investigational Drugs (1999), 1(4), 466-469; Muzina, D. J., El-Sayegh, S., Calabrese, J. R., Antiepileptic drugs in psychiatry-focus on randomized controlled trial, Epilepsy Research, 2002, 50 (1-2), 195-202; and Calabrese, J. R., Shelton, M. D., Rapport, D. J., Kimmel, S. E., Bipolar disorders and the effectiveness of novel anticonvulsants, J. Clin. Psychiatry, 2002, 63 (suppl 3), 5-9.

Compounds of the present invention can be used to treat psychiatric disorders including, but not limited to, anxiety and panic disorders, post-traumatic stress disorder, schizophrenia, episodic dyscontrol, substance-abuse-related disorders, impulse control disorders, general agitation associated with a variety of psychiatric disorders and dementias, and behavioral disorders associated with autism as described in Bialer, M., Johannessen, S. I., Kupferberg, H. J., Levy, R. H., Loiseau, P., Perucca, E., Progress report on new antiepileptic drugs: a summary of the sixth eilat conference (EILAT VI), Epilepsy Res. 2002, 51, 31-71; Fountain, N. B., Dreifuss, F. E., The future of valproate. In: Valproate., Löscher W., Editor. 1999, Birkhauser Verlag, Boston; Fountain, N. B., Dreifuss, F. E., The future of valproate. In: Valproate., Löscher W., Editor. 1999, Birkhauser Verlag, Boston; and Balfour, J. A., Bryson, H. M. Valproic acid: A review of its pharmacology and therapeutic potential in indications other than epilepsy, CNS Drugs, 1994, 2 (2), 144-173.

Compounds of the present can be used to treat different types of migraine such as classical migraine and common migraine as described in Wheeler, S. D., Antiepileptic drugs therapy in migraine headache, Current Treatment Options in Neurology, 2002, 4, 383-394; and Krymchantowski, A. V., Bigal, M. E., Moreira, P. E., New and emerging prophylactic agents for migraine, CNS Drugs, 2002, 16 (9), 611-634.

Compounds of the present invention can be used to treat pain including, but not limited to, neuropathic pain including, but no limited to, diabetic neuropathy, cancer neuropathy, HIV pain, trigeminal neuralgia, post-herpetic neuralgia, traumatic neuralgia, phantom limb, severe refractory pain, and lancinating pain as described in Tremont-Lukats, I. W., Megeff, C., Backonja, M.-M., Anticonvulsants for neuropathich pain syndromes: mechanisms of action and place in therapy, Drugs, 60 (5), 1029-1052; Jensen, T. S., Anticonvulsants in neuropathic pain: rationale and clinical evidence, Eur. J. Pain, 2002, 6 (suppl A), 61-68; and Balfour, J. A., Bryson, H. M. Valproic acid: A review of its pharmacology and therapeutic potential in indications other than epilepsy, CNS Drugs, 1994, 2 (2), 144-173; Hardy, J. R., Rees, E. A. J., Gwilliam, B., Ling, J., Broadley, K., A'Hern, R., J. of Pain and Symptom Management, 2001, 21 (3), 204-209.

Compounds of the present invention can be used to provide neuroprotection as described in Pitkanen, A., Efficacy of current antiepileptics to prevent neurodegeneration in epilepsy models, Epilepsy Research, 2002, 50, 141-160.

Compounds of the present invention can be used to treat movement disorders including, but not limited to, restless leg syndrome, periodic limb movements of sleep, essential tremor, acquired nystagmus, post-anoxic myoclonus, spinal myoclonus, spasticity, chorea, and dystonia as described in Magnus, L., Nonepileptic uses of gabapentin, Epilepsia, 1999, 40 (suppl 6), S66-S72; Fountain, N. B., Dreifuss, F. E., The future of valproate. In: Valproate., Löscher W., Editor. 1999, Birkhauser Verlag, Boston; Cutter, N., Scott, D. D., Johnson, J. C., Whiteneck, G., Gabapentin effect on spacticity in multiple sclerosis, 2000, 81, 164-169.

The present invention also provides pharmaceutical compositions that comprise compounds of the present invention. The pharmaceutical compositions comprise compounds of the present invention formulated together with one or more non-toxic pharmaceutically acceptable carriers.

The pharmaceutical compositions of this invention can be administered to humans and other mammals orally, rectally, parenterally, intracisternally, intravaginally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable carrier or excipient, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of this invention include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like. Prodrugs of the present invention may be rapidly transformed in vivo to compounds of formula (I), for example, by hydrolysis in blood.

The present invention contemplates compounds of formula (I) formed by synthetic means or formed by in vivo biotransformation.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The total daily dose of the compounds of this invention administered to a human or lower animal may range from about 0.003 to about 90 mg/kg/day. For purposes of oral administration, more preferable doses can be in the range of from about 0.01 to about 30 mg/kg/day. If desired, the effective daily dose can be divided into multiple doses for purposes of administration; consequently, single dose compositions may contain such amounts or submultiples thereof to make up the daily dose.

What is claimed is:

1. A method of treating migraine, epilepsy, or bipolar disorder in a mammal comprising administering to a mammal a therapeutically effective amount of a compound of formula (I) 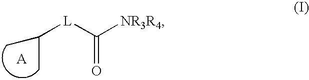
or a pharmaceutically acceptable prodrug thereof, wherein
A is cycloalkyl or bridged cycloalkyl;
L is a single bond or alkylene;
R₃ and R₄ are independently hydrogen, alkenyl, alkyl, alkynyl, alkoxycarbonylalkyl, aryl, arylalkyl, carboxyalkyl, cycloalkyl, cycloalkylalkyl, heterocycle, heterocyclealkyl, hydroxyalkyl, (NR₅R₆)alkyl, (NR₅R₆) carbonylalkyl, or 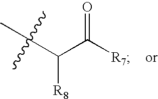
R₃ and R₄ taken together with the nitrogen atom to which they are attached form a heterocycle wherein the heterocycle is azepanyl, azetidinyl, aziridinyl, morpholinyl, piperazinyl, piperidinyl, pyrrolidinyl, or thiomorpholinyl;
R₅ and R₆ are independently hydrogen, alkenyl, alkyl, alkynyl, alkoxycarbonylalkyl, aryl, arylalkyl, cycloalkyl, cycloalkylalkyl, heterocycle, heterocyclealkyl, or hydroxyalkyl;
R₇ is alkoxy, alkyl, hydroxy, or —NR₅R₆;
R₈ is alkenyl, alkoxyalkyl, alkoxycarbonylalkyl, alkylthioalkyl, alkynyl, aryl, arylalkyl, carboxyalkyl, cycloalkyl, cycloalkylalkyl, heterocycle, heterocyclealkyl, hydroxyalkyl, mercaptoalkyl, (NR₅R₆)alkyl, (NR₅R₆)carbonylalkyl, or —(CH₂)ₙNHC(═NH)NH₂; and
n is an integer from 1 to 6;
provided that the compound of formula (I) is other than cyclohexanecarboxamide.

2. The method according to claim 1 wherein A is cycloalkyl.

3. The method according to claim 1 wherein
A is cycloalkyl wherein the cycloalkyl is cyclohexyl optionally substituted with 1, 2, or 3 alkyl groups;
L is a single bond; and
R₃ and R₄ are hydrogen.

4. The method according to claim 1 wherein
A is cycloalkyl wherein the cycloalkyl is cyclohexyl substituted with 1, 2, or 3 alkyl groups;
L is a single bond; and
R₃ and R₄ are hydrogen.

5. The method according to claim 4 wherein the compound of formula (I) is
(cis) (3R,5S)-3,5-dimethylcyclohexanecarboxamide;
2,3-dimethylcyclohexanecarboxamide;
4-methylcyclohexanecarboxamide;
3-methylcyclohexanecarboxamide;
2-methylcyclohexanecarboxamide;
2,5-dimethylcyclohexanecarboxamide;
3,4-dimethylcyclohexanecarboxamide;
4-isopropylcyclohexanecarboxamide;
4-tert-butylcyclohexanecarboxamide;
2,4-dimethylcyclohexanecarboxamide; or
(cis) (2R,6S)-2,4,6-trimethylcyclohexanecarboxamide.

6. The method according to claim 1 wherein
A is cycloalkyl wherein the cycloalkyl is cyclopentyl optionally substituted with 1, 2, or 3 alkyl substituents;
L is a single bond; and
R₃ and R₄ are hydrogen.

7. The method according to claim 6 wherein the compound of formula (I) is cyclopentanecarboxamide.

8. The method according to claim 1 wherein
A is cycloalkyl wherein the cycloalkyl is cyclohexyl optionally substituted with 1, 2, or 3 alkyl groups;
L is a single bond;
R₃ is hydrogen;
R₄ is (NR₅R₆)carbonylalkyl; and
R₅ and R₆ are hydrogen.

9. The method according to claim 1 wherein
A is cycloalkyl wherein the cycloalkyl is cyclohexyl substituted with 1, 2, or 3 alkyl groups;
L is a single bond;
R₃ is hydrogen;
R₄ is (NR₅R₆) carbonylalkyl; and
R₅ and R₆ are hydrogen.

10. The method according to claim 9 wherein the compound of formula (I) is
N-(2-amino-2-oxoethyl)-4-methylcyclohexanecarboxamide;
N-(2-amino-2-oxoethyl)-3 -methylcyclohexanecarboxamide;
(1S,2R,5S)—N-(2-amino-2-oxoethyl)-2,5-dimethylcyclohexanecarboxamide;
N-(2-amino-2-oxoethyl)-2,3-dimethylcyclohexanecarboxamide;
N-(2-amino-2-oxoethyl)-2-methylcyclohexanecarboxamide;
N-(2-amino-2-oxoethyl)-3,4-dimethylcyclohexanecarboxamide;
N-(2-amino-2-oxoethyl)-4-isopropylcyclohexanecarboxamide;
N-(2-amino-2-oxoethyl)-4-tert-butylcyclohexanecarboxamide;
N-(2-amino-2-oxoethyl)-2,4-dimethylcyclohexanecarboxamide;
(cis) (3R,5S)—N-[(1S)-2-amino-1-methyl-2-oxoethyl]-3,5-dimethylcyclohexanecarboxamide;
(cis) (3R,5S)—N-(3-amino-3-oxopropyl)-3,5-dimethylcyclohexanecarboxamide;
(cis) (3R,5S)—N-[(1R)-2-amino-1-methyl-2-oxoethyl]-3,5-dimethylcyclohexanecarboxamide;
(cis) (3R,5S)—N-[(1S)-1-(aminocarbonyl)-2-methylpropyl]-3,5-dimethylcyclohexanecarboxamide;
N-(2-amino-2-oxoethyl)-2,5-dimethylcyclohexanecarboxamide; or
(cis) (2R,6S)—N-(2-amino-2-oxoethyl)-2,4,6-trimethylcyclohexanecarboxamide.

11. The method according to claim 9 wherein the compound of formula (I) is (cis) (3R,5S)—N-(2-amino-2-oxoethyl)-3,5-dimethylcyclohexanecarboxamide.

12. The method according to claim 1 wherein
A is cycloalkyl wherein the cycloalkyl is cyclopentyl optionally substituted with 1, 2, or 3 alkyl groups;
L is a single bond;
R₃ is hydrogen;
R₄ is (NR₅R₆)carbonylalkyl; and
R₅ and R₆ are hydrogen.

13. The method according to claim 12 wherein the compound of formula (I) is N-(2-amino-2-oxoethyl)cyclopentanecarboxamide.

14. The method according to claim 1 wherein
A is cycloalkyl wherein the cycloalkyl is cyclohexyl optionally substituted with 1, 2, or 3 alkyl groups;
L is a single bond;
R₃ is hydrogen; and
R₄ is

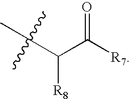

15. The method according to claim 1 wherein
A is cycloalkyl wherein the cycloalkyl is cyclohexyl optionally substituted with 1, 2, or 3 alkyl groups;
L is a single bond;
R₃ is hydrogen;
R₄ is 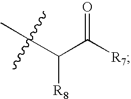
R₇ is NR₅R₆;
R₅ and R₆ are hydrogen; and
R₈ is heterocycle wherein the heterocycle is imidazolyl.

16. The method according to claim 1 wherein
A is cycloalkyl wherein the cycloalkyl is cyclohexyl substituted with 1, 2, or 3 alkyl groups;
L is a single bond;
R₃ is hydrogen;
R₄is 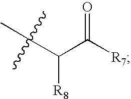
R₇ is NR₅R₆;
R₅ and R₆ are hydrogen; and
R₈ is heterocycle wherein the heterocycle is imidazolyl.

17. The method according to claim 16 wherein the compound of formula (I) is (cis) (3R,5S)—N-[(1S)-2-amino-1-(1H-imidazol-4-ylmethyl)-2-oxoethyl]-3,5-dimethylcyclohexanecarboxamide.

18. The method according to claim 1 wherein
A is cycloalkyl wherein the cycloalkyl is cyclohexyl optionally substituted with 1, 2, or 3 alkyl groups;
L is a single bond;
R₃ is hydrogen; and
R₄ is carboxyalkyl.

19. The method according to claim 1 wherein
A is cycloalkyl wherein the cycloalkyl is cyclohexyl substituted with 1, 2, or 3 alkyl groups;
L is a single bond;
R₃ is hydrogen; and
R₄ is carboxyalkyl.

20. The method according to claim 19 wherein the compound of formula (I) is (cis) (2S)-2-({[(3R,5S)-3,5-dimethylcyclohexyl]carbonyl}amino)propanoic acid.

21. The method according to claim 1 wherein
A is cycloalkyl wherein the cycloalkyl is cyclohexyl optionally substituted with 1, 2, or 3 alkyl groups;
L is a single bond;
R₃ is hydrogen; and
R₄ is hydroxyalkyl.

22. The method according to claim 1 wherein
A is cycloalkyl wherein the cycloalkyl is cyclohexyl substituted with 1, 2, or 3 alkyl groups;
L is a single bond;
R₃ is hydrogen; and
R₄ is hydroxyalkyl.

23. The method according to claim 22 wherein the compound of formula (I) is
(cis) (3R,5S)—N-[(2R)-2-hydroxypropyl]-3,5-dimethylcyclohexanecarboxamide; or
(cis) (3R,5S)—N-[(2S)-2-hydroxypropyl]-3,5-dimethylcyclohexanecarboxamide.

24. The method according to claim 1 wherein
A is cycloalkyl wherein the cycloalkyl is cyclopropyl optionally substituted with 1 or 2 cyclopropyl groups;
L is a single bond;
R₃ is hydrogen;
R₄ is hydrogen or (NR₅R₆)carbonylalkyl; and
R₅ and R₆ are hydrogen.

25. The method according to claim 1 wherein
A is cycloalkyl wherein the cycloalkyl is cyclopropyl substituted with 2 cyclopropyl groups;
L is a single bond;
R₃ is hydrogen;
R₄ is hydrogen or (NR₅R₆)carbonylalkyl; and
R₅ and R₆ are hydrogen.

26. The method according to claim 25 wherein the compound of formula (I) is
1,1′:1′1″-ter(cyclopropane)-2′-carboxamide; or
N-(2-amino-2-oxoethyl)-1,1′:1′,1″-ter(cyclopropane)-2′-carboxamide.

27. The method according to claim 1 wherein
A is cycloalkyl wherein the cycloalkyl is cyclohexyl optionally substituted with 1, 2, or 3 alkyl groups;
L is alkylene wherein the alkylene is CH₂; and
R₃ and R₄ are hydrogen.

28. The method according to claim 27 wherein the compound of formula (I) is 2-cyclohexylacetamide.

29. The method according to claim 1 wherein
A is cycloalkyl wherein the cycloalkyl is cyclohexyl substituted with 1, 2, or 3 alkyl groups;
L is alkylene wherein the alkylene is CH₂; and
R₃ and R₄ are hydrogen.

30. The method according to claim 29 wherein the compound of formula (I) is
2-(3-methylcyclohexyl)acetamide;
2-(4-methylcyclohexyl)acetamide;
2-(2-methylcyclohexyl)acetamide;
2-(5-isopropyl-2-methylcyclohexyl)acetamide;
2-(4-tert-butylcyclohexyl)acetamide; or
2-(4,4-dimethylcyclohexyl)acetamide.

31. The method according to claim 1 wherein
A is cycloalkyl wherein the cycloalkyl is cyclopentyl optionally substituted with 1, 2, or 3 alkyl groups;
L is alkylene wherein the alkylene is CH₂; and
R₃ and R₄ are hydrogen.

32. The method according to claim 31 wherein the compound of formula (I) is 2-cyclopentylacetamide.

33. The method according to claim 1 wherein
A is cycloalkyl wherein the cycloalkyl is cyclohexyl optionally substituted with 1, 2, or 3 alkyl groups;
L is alkylene wherein the alkylene is CH₂;
R₃ is hydrogen;
R₄ is (NR₅R₆)carbonylalkyl; and
R₅ and R₆ are hydrogen.

34. The method according to claim 33 wherein the compound of formula (I) is N-(2-amino-2-oxoethyl)-2-cyclohexylacetamide.

35. The method according to claim 1 wherein
A is cycloalkyl wherein the cycloalkyl is cyclohexyl substituted with 1, 2, or 3 alkyl groups;
L is alkylene wherein the alkylene is CH₂;
R₃ is hydrogen;
R₄ is (NR₅R₆)carbonylalkyl; and
R₅ and R₆ are hydrogen.

36. The method according to claim 35 wherein the compound of formula (I) is
N-(2-amino-2-oxoethyl)-2-(3-methylcyclohexyl)acetamide;
N-(2-amino-2-oxoethyl)-2-(4-methylcyclohexyl)acetamide;
N-(2-amino-2-oxoethyl)-2-(2-methylcyclohexyl)acetamide;
N-(2-amino-2-oxoethyl)-2-(5-isopropyl-2-methylcyclohexyl)acetamide;
N-(2-amino-2-oxoethyl)-2-(4-tert-butylcyclohexyl)acetamide; or
N-(2-amino-2-oxoethyl)-2-(4,4-dimethylcyclohexyl)acetamide.

37. The method according to claim 1 wherein
A is cycloalkyl wherein the cycloalkyl is cyclopentyl optionally substituted with 1, 2, or 3 alkyl groups;
L is alkylene wherein the alkylene is CH₂;
R₃ is hydrogen;
R₄ is (NR₅R₆)carbonylalkyl; and
R₅ and R₆ are hydrogen.

38. The method according to claim 37 wherein the compound of formula (I) is N-(2-amino-2-oxoethyl)-2-cyclopentylacetamide.

39. The method according to claim 1 wherein A is bridged cycloalkyl.

40. The method according to claim 1 wherein
A is bridged cycloalkyl wherein the bridged cycloalkyl is adamantane, bicyclo[2.2.1]heptane, or octahydro-2,5-methanopentalene;
L is a single bond; and
R₃ and R₄ are hydrogen.

41. The method according to claim 23 wherein the compound of formula (I) is
bicyclo[2.2.1]heptane-2-carboxamide;
1-adamantanecarboxamide; or
hexahydro-2,5-methanopentalene-3a(1H)-carboxamide.

42. The method according to claim 1 wherein
A is bridged cycloalkyl wherein the bridged cycloalkyl is adamantane, bicyclo[2.2.1]heptane, or octahydro-2,5-methanopentalene;
L is a single bond;
R₃ is hydrogen;
R₄ is (NR₅R₆)carbonylalkyl; and
R₅ and R₆ are hydrogen.

43. The method according to claim 42 wherein the compound of formula (I) is
N-(2-amino-2-oxoethyl)bicyclo[2.2.1]heptane-2-carboxamide;
N-(2-amino-2-oxoethyl)-1-adamantanecarboxamide; or
N-(2-amino-2-oxoethyl)hexahydro-2,5-methanopentalene-3a(1H)-carboxamide.

44. The method according to claim 1 wherein
A is bridged cycloalkyl wherein the bridged cycloalkyl is adamantane, bicyclo[2.2.1]heptane, or octahydro-2,5-methanopentalene;
L is alkylene wherein the alkylene is CH₂;
R₃ is hydrogen;
R₄ is hydrogen or (NR₅R₆)carbonylalkyl; and
R₅ and R₆ are hydrogen.

45. The method according to claim 44 wherein the compound of formula (I) is
2-bicyclo[2.2.1]hept-2-ylacetamide;
N-(2-amino-2-oxoethyl)-2-bicyclo[2.2.1]hept-2-ylacetamide;
2-(1-adamantyl)acetamide; or
2-(1-adamantyl)-N-(2-amino-2-oxoethyl)acetamide.

46. A method of treating a psychiatric disorder, pain, or a movement disorder in a mammal comprising administering to a mammal a therapeutically effective amount of a compound of formula (I).

47. The method according to claim 46 wherein the compound of formula (I) is (cis) (3R,5S)—N-(2-amino-2-oxoethyl)-3,5-dimethylcyclohexanecarboxamide.

48. A method of providing neuroprotection in a mammal comprising administering to a mammal a therapeutically effective amount of a compound of formula (I).

49. The method according to claim 48 wherein the compound of formula (I) is (cis) (3R,5S)—N-(2-amino-2-oxoethyl)-3,5-dimethylcyclohexanecarboxamide.

50. A compound of formula (II) 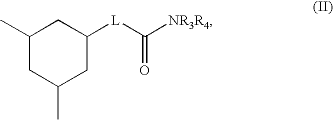
or a pharmaceutically acceptable prodrug thereof, wherein
L is a single bond or alkylene;
R₃ is hydrogen or alkyl;
R₄ is alkenyl, alkyl, alkynyl, alkoxycarbonylalkyl, aryl, arylalkyl, carboxyalkyl, cycloalkyl, cycloalkylalkyl, heterocycle, heterocyclealkyl, hydroxyalkyl, (NR₅R₆)alkyl, (NR₅R₆)carbonylalkyl, or 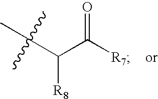
R₃ and R₄ taken together with the nitrogen atom to which they are attached form a heterocycle wherein the heterocycle is azepanyl, azetidinyl, aziridinyl, morpholinyl, piperazinyl, piperidinyl, pyrrolidinyl, or thiomorpholinyl;
R₅ and R₆ are independently hydrogen, alkenyl, alkyl, alkynyl, alkoxycarbonylalkyl, aryl, arylalkyl, cycloalkyl, cycloalkylalkyl, heterocycle, heterocyclealkyl, or hydroxyalkyl;
R₇ is alkoxy, alkyl, hydroxy, or —NR₅R₆;
R₈ is alkenyl, alkoxyalkyl, alkoxycarbonylalkyl, alkylthioalkyl, alkynyl, aryl, arylalkyl, carboxyalkyl, cycloalkyl, cycloalkylalkyl, heterocycle, heterocyclealkyl, hydroxyalkyl, mercaptoalkyl, (NR₅R₆)alkyl, (NR₅R₆)carbonylalkyl, or —(CH₂)ₙNHC(═NH)NH₂; and
n is an integer from 1 to 6.

51. The compound according to claim 50 wherein
R₃ is hydrogen; and
R₄ is (NR₅R₆)carbonylalkyl.

52. The compound according to claim 50 wherein
L is a single bond;
R₃ is hydrogen;
R₄ is (NR₅R₆)carbonylalkyl; and
R₅ and R₆ are hydrogen.

53. The compound according to claim 52 wherein the compound of formula (II) is
(cis) (3R,5S)—N-[(1S)-2-amino-1-methyl-2-oxoethyl]-3,5-dimethylcyclohexanecarboxamide;
(cis) (3R,5S)—N-(3-amino-3-oxopropyl)-3,5-dimethylcyclohexanecarboxamide;
(cis) (3R,5S)—N-[(1R)-2-amino-1-methyl-2-oxoethyl]-3,5-dimethylcyclohexanecarboxamide; or
(cis) (3R,5S)—N-[(1S)-1-(aminocarbonyl)-2-methylpropyl]-3,5-dimethylcyclohexanecarboxamide.

54. The compound according to claim 52 wherein the compound of formula (II) is (cis) (3R,5S)—N-(2-amino-2-oxoethyl)-3,5-dimethylcyclohexanecarboxamide.

55. The compound according to claim 50 wherein
L is a single bond;
R₃ is hydrogen; and
R₄ is carboxyalkyl.

56. The compound according to claim 55 wherein the compound of formula (II) is (cis) (2S)-2-({[(3R,5S)-3,5-dimethylcyclohexyl]carbonyl}amino)propanoic acid.

57. The compound according to claim 50 wherein
L is a single bond;
R₃ is hydrogen; and
R₄ is

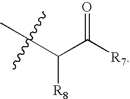

58. The compound according to claim 50 wherein
L is a single bond;
R₃ is hydrogen;
R₄ is 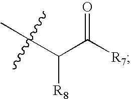
R₇ is —NR₅R₆;
R₅ and R₆ are independently hydrogen or alkyl; and
R₈ is heterocycle wherein the heterocycle is imidazolyl.

59. The compound according to claim 58 wherein the compound of formula (II) is (cis) (3R,5S)—N-[(1S)-2-amino-1-(1H-imidazol-4-ylmethyl)-2-oxoethyl]-3,5-dimethylcyclohexanecarboxamide.

60. The compound according to claim 50 wherein
L is a single bond;
R₃ is hydrogen; and
R₄ is hydroxyalkyl.

61. The compound according to claim 60 wherein the compound of formula (II) is
(cis) (3R,5S)—N-[(2R)-2-hydroxypropyl]-3,5-dimethylcyclohexanecarboxamide; or
(cis) (3R,5S)—N-[(2S)-2-hydroxypropyl]-3,5-dimethylcyclohexanecarboxamide.

62. A method of treating neuropathic and inflammatory pain in a mammal comprising administering to a mammal a therapeutically effective amount of a compound of formula (I).
